(19) United States
(12) Patent Application Publication
(10) Pub. No.: US 2023/0310601 A1
(43) Pub. Date: Oct. 5, 2023

(54) CHIMERIC ANTIGEN RECEPTOR-MODIFIED IMMUNE CELLS EXPRESSING A SECRETABLE BISPECIFIC TRAP PROTEIN AND USES THEREOF

(71) Applicant: University of Southern California, Los Angeles, CA (US)

(72) Inventors: Xianhui CHEN, Los Angeles, CA (US); Pin WANG, Los Angeles, CA (US)

(73) Assignee: University of Southern California, Los Angeles, CA (US)

(21) Appl. No.: 18/192,997

(22) Filed: Mar. 30, 2023

Related U.S. Application Data
(60) Provisional application No. 63/325,225, filed on Mar. 30, 2022.

(51) Int. Cl. A61K 39/00; C07K 16/28; C07K 14/71; C07K 14/705; C07K 14/725; A61P 35/00

(52) U.S. Cl. CPC A61K 39/4611; C07K 16/2803; C07K 16/2818; C07K 14/71; C07K 14/70521; C07K 14/7051; A61P 35/00; A61K 39/4631; A61K 39/4633; A61K 39/4643; C07K 2319/02; C07K 2317/622; C07K 2319/03; A61K 2239/13; A61K 2239/21; A61K 2239/22; A61K 2239/29

(57) ABSTRACT

Herein, we provide genetically engineered immune effector cells, among other cells, which express CAR and secret a bispecific “trap” protein co-targeting a checkpoint protein and TGF-β or TGF-β receptor, so as to improve the antitumor immunity of the immune effector cells. Compared with conventional CAR-T cells and CAR-T cells secreting a polypeptide checkpoint inhibitor, the provided genetically engineered immune effector cells CAR-T cells with “trap” protein secretion attenuate inhibitory T cell signaling, enhance T cell persistence and expansion, and improve effector functionalities and resistance to exhaustion. In a xenograft mouse model, CAR-T cells with “trap” protein secretion significantly enhanced antitumor immunity and efficacy. Methods of using these genetically engineered cells, as well as using polynucleotides encoding the CAR and the “trap” protein, are also provided, for example, as a therapy against solid tumors.

### CROSS-REFERENCE TO RELATED APPLICATIONS

This application includes a claim of priority under 35 U.S.C. § 119(e) to U.S. provisional patent application No. 63/325,225, filed Mar. 30, 2022, the entirety of which is hereby incorporated by reference.

### STATEMENT REGARDING FEDERALLY SPONSORED RESEARCH OR DEVELOPMENT

This invention was made with government support under Grant Nos. A1068978, CA170820, EB017206, and CA132681 awarded by the National Institutes of Health (NIH). The government has certain rights in the invention.

### REFERENCE TO SEQUENCE LISTING

This application contains a Sequence Listing submitted as an electronic file named “065715_000114USPT_SequenceListing.xml”, having a size in bytes of 28,286 bytes, and created on Mar. 29, 2023 (production date noted as 2023-03-30). The information contained in this electronic file is hereby incorporated by reference in its entirety.

### FIELD OF INVENTION

This invention relates to cell therapies improved with secretable bispecific proteins to overcome the inhibitory effect in the immunosuppressive tumor microenvironment.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Over the last two decades, adoptive transfer of chimeric antigen receptor-engineered T (CAR-T) cells has emerged as a promising therapeutic strategy for management and treatment of cancer. In general, CARs are synthetic proteins expressed on T cell surface, consisting of an extracellular antigen binding domain, a hinge, a transmembrane domain, and intracellular costimulatory domain and activation domain. Upon antigen recognition, CAR-T cells can be activated and exhibit MHC-unrestricted tumor cell killing effect. The potential of CAR-T therapy for hematological malignancies has been validated by clinical studies. The two anti-CD19 CAR-T therapies approved by FDA encouraged more research aimed at developing CAR-T therapies with higher remission rates in more cancer types. However, despite extensive efforts, the success of CAR-T therapy is not yet extrapolated to solid tumors.

Tumor microenvironment (TME) in solid tumor imposes immunosuppression to CAR-T cells, constituting a major challenge to the success of CAR-T therapy in solid tumors. Besides the physical and metabolic barriers (e.g., low oxygen, low nutrient, low pH), multiple mechanisms in TME act to inhibit CAR-T cell function and expansion. For instance, tumor cells have upregulated expression of immune checkpoint ligands such as programmed cell death ligand 1 (PD-L1). When PD-L1 bind to its receptor programmed cell death protein 1 (PD-1) on CAR-T cells, the immune checkpoint interaction activates immunosuppressive cell signaling that causes CAR-T cell dysfunction and exhaustion, ultimately leading to the immune tolerance of tumor cells. Recent preclinical and clinical studies have shown that knockdown or knockout of the PD-1 gene in CAR-T cells, or combining immune checkpoint blockades with CAR-T cells, could significantly augment T cell immune response and enhance the antitumor efficacy of CAR-T therapy.

Another well-defined immunosuppressive mechanism in the TME comes from the soluble molecules secreted by tumor cells, stromal cells and suppressive immune cells. Among these molecules, transforming growth factor β (TGF-β) is particularly important in inhibiting T cell effector function and inducing T cell differentiation into the regulatory phenotype. Immunosuppression from TGF-β is potent and associated with immune-checkpoint signaling pathways. Studies have found that active TGF-β signaling in TME might be responsible for the poor response rates observed in clinical trials of checkpoint inhibitors, especially in the treatment for prostate cancer, ovarian cancer, and breast cancer.

Therefore, it is an objective of the present invention to provide a new approach for combinational CAR-T therapy. It is another objective of the present invention to improve the therapeutic efficacy of CAR-T therapy by reducing the amount and/or activity of immune suppressive molecules and immune checkpoint proteins.

### SUMMARY OF THE INVENTION

The following embodiments and aspects thereof are described and illustrated in conjunction with compositions and methods which are meant to be exemplary and illustrative, not limiting in scope.

Various embodiments provide genetically engineered cells, preferably genetically engineered immune cells or immune effector cells, wherein the genetically engineered cells comprise one or more polynucleotides encoding: a) a chimeric antigen receptor (CAR); b) a polypeptide that binds an immune checkpoint protein or a polypeptide that binds a ligand of the immune checkpoint protein, also referred to as a “polypeptide checkpoint inhibitor”; and c) a polypeptide that binds transforming growth factor beta (TGF-β) or a polypeptide that binds a TGF-receptor, also referred to as a “polypeptide binder of TGF-β or TGF-β receptor,” respectively.

Various embodiments provide genetically engineered cells, which contains the one or more polynucleotides that further encode d) a signal peptide, so as to augment the secretion rates and/or serum (or culture medium) level of the secreted protein(s), e.g., b) the polypeptide checkpoint inhibitor and c) the polypeptide binder of TGF-β or TGF-β receptor.

In some embodiments, a genetically engineered cell includes one polynucleotide that encodes a) a CAR, b) a polypeptide that binds an immune checkpoint protein or a polypeptide that binds a ligand of the immune checkpoint protein, and c) a polypeptide that binds TGF-β or a polypeptide that binds a TGF-β receptor. In further embodiments, the one polynucleotide further encodes a cleavable peptide in a configuration wherein a) the CAR is operably linked via the cleavable peptide to the 5′ or 3′ end of b) the polypeptide that binds an immune checkpoint protein or the polypeptide that binds a ligand of the immune checkpoint protein, or to the 5′ or 3′ end of c) the polypeptide that binds TGF-β or the polypeptide that binds a TGF-β receptor. In some embodiments, a cleavable peptide is a 2A peptide, e.g., self-cleaving, and exemplary 2A peptide comprises a T2A peptide, a P2A peptide, an E2A peptide, an F2A peptide, or a combination thereof. In other embodiments, a cleavable peptide is a protease-sensitive peptide. In further embodiments, the one polynucleotide further encodes a signal peptide located at the N-terminus of b) the polypeptide checkpoint inhibitor and c) the polypeptide binder of TGF-β or TGF-β receptor.

In some embodiments, a genetically engineered cell includes at least two polynucleotides, wherein a first of the two polynucleotides encodes a) the CAR, and a second of the two polypeptides encodes b) the polypeptide that binds an immune checkpoint protein or the polypeptide that binds a ligand of the immune checkpoint protein and c) the polypeptide that binds TGF-β or the polypeptide that binds a TGF-β receptor. In further embodiments, at least one of the two polynucleotides further encodes d) a signal peptide at the N-terminus. In some embodiments, the second of the two polypeptides further encodes d) a signal peptide. In other embodiments, a genetically engineered cell includes a third polynucleotide that encodes d) a signal peptide.

Preferably, the polynucleotide encoding at least b) the polypeptide that binds an immune checkpoint protein or the polypeptide that binds a ligand of the immune checkpoint protein and c) the polypeptide that binds TGF-β or the polypeptide that binds a TGF-β receptor, upon expression, is transcribed to a fusion protein comprising b) the polypeptide that binds an immune checkpoint protein or the polypeptide that binds a ligand of the immune checkpoint protein and c) the polypeptide that binds TGF-β or the polypeptide that binds a TGF-β receptor, upon expression. Optionally the fusion protein further includes a linker between b) the polypeptide that binds an immune checkpoint protein or the polypeptide that binds a ligand of the immune checkpoint protein and c) the polypeptide that binds TGF-β or the polypeptide that binds a TGF-β receptor, upon expression. In further embodiments, the linker in the fusion is not a self-cleaving peptide or a protease cleavable peptide, or the linker in the fusion protein is not degradable in a tumor microenvironment or in contact with tumor cells. Preferably, a signal peptide is at the N-terminus of the fusion protein.

Further embodiments provide that genetically engineered cells disclosed herein secrete the fusion protein comprising the polypeptide that binds an immune checkpoint protein and the polypeptide that binds TGF-β. In some embodiments, the fusion protein further comprises a signal protein, preferably located at the N-terminus.

In some embodiments, genetically engineered cells express, or include a polynucleotide encoding, a polypeptide that binds an immune checkpoint protein and the polypeptide that binds TGF-β.

In some embodiments, the polypeptide that binds an immune checkpoint protein comprises a single-chain variable fragment (scFv), a single monomeric variable domain, or an antigen-binding fragment of an anti-programmed cell death protein 1 (PD-1) antibody, an anti-lymphocyte-activation gene 3 (LAG3) antibody, an anti-T cell immunoglobulin domain and mucin domain-containing protein 3 (TIM3) antibody, an anti-T-lymphocyte antigen-4 (CTLA-4) antibody, or a combination thereof; the polypeptide that binds a ligand of the immune checkpoint protein comprises a single-chain variable fragment (scFv), a single monomeric variable domain, or an antigen-binding fragment of an anti-PD-1 ligand (PD-L1) antibody.

In some embodiments, the polypeptide that binds an immune checkpoint protein is a polypeptide that binds PD-1, and the polypeptide that binds PD-1 comprises a single-chain variable fragment (scFv) a single monomeric variable domain, or a PD-1-binding fragment of one or more of nivolumab, pembrolizumab, cemiplimab, dostarlimab, vopratelimab, spartalizumab, camrelizumab, sintilimab, tislelizumab, toripalimab, INCMGA00012, AMP-224, AMP-514, and acrixolimab.

In some embodiments, a polypeptide that binds TGF-β comprises a TGF-β receptor II ectodomain sequence.

In some embodiments, the signal peptide is an interleukin-2 (IL-2) signal peptide or a variant thereof, wherein the variant is at least 95%, 90%, 85%, or 80% sequence identical to the wild-type IL-2 signal peptide and, when expressed at the N-terminus of a protein, results in at least 80%, 90%, 100%, 110%, 120% or more of the secretion level of the protein, compared to when the wild-type IL-2 signal peptide is expressed at the N-terminus of the protein.

In various embodiments, the genetically engineered cells comprise a T-lymphocyte (T-cell), a natural killer (NK) cell, a hematopoietic stem cell (HSC), an embryonic stem cell (ESC), a pluripotent stem cell, or a combination thereof. In some embodiments, the T-cells, NK cells, HSCs, and/or pluripotent stem cells are obtained from a subject, wherein these cells after the genetic engineering disclosed herein are infused or administered back to the subject.

In some embodiments, a composition is provided which includes a plurality of the genetically engineered cells, wherein at least 50%, 60%, 70%, 80%, or 90% or all of the plurality of the genetically engineered cells express the CAR. In some embodiments, transducing a population of T-cells, NK cells, HSCs, ESCs, and/or pluripotent stem cells with a polynucleotide encoding the three elements a)-c) or the four elements a)-d) disclosed herein may result in at least 50%, 60%, 70%, 80%, or 90% of the population of cells expressing a) the CAR and secreting a fusion protein comprising the b) and c) elements. In further embodiments, at least 10%, 20%, 30%, 40%, or 50% of the population of the genetically engineered cells maintain expression of the CAR after at least one freeze-and-thaw cycle.

Various embodiments further provide an isolated nucleic acid, e.g., a polynucleotide, which encodes: (i) a fusion protein comprising a polypeptide checkpoint inhibitor and a polypeptide binder of transforming growth factor beta (TGF-β), and (ii) a chimeric antigen receptor (CAR). In some embodiments, wherein the CAR and the fusion protein are operably linked by a cleavable peptide linker. In some embodiments, the polypeptide checkpoint inhibitor comprises an antigen-binding fragment of one or more of an anti-programmed cell death protein 1 (PD-1) antibody, an anti-lymphocyte-activation gene 3 (LAG3) antibody, an anti-T cell immunoglobulin domain and mucin domain-containing protein 3 (TIM3) antibody, and an anti-T-lymphocyte antigen-4 (CTLA-4) antibody. In some embodiments, the polypeptide binder of TGF-comprises a TGF-β-binding fragment of a TGF-β receptor or a TGF-β-binding fragment of an anti-TGF-β antibody.

In some embodiments, a polynucleotide is provided, which encodes the following elements: a cleavable peptide linker comprising a T2A sequence; a polypeptide checkpoint inhibitor comprising a single-chain variable fragment (scFv) of an anti-PD-1 antibody or a fragment thereof being a single-domain antibody; and the polypeptide binder of TGF-β comprises amino acid sequence of a ligand binding region in human TGF-βRII extracellular domain. In some embodiments, a polynucleotide is provided, which from 5′ to 3′ end encodes: a CAR, a T2A sequence, a human IL-2 leading sequence, a light chain variable domain of the anti-PD-1 antibody, a first peptide linker having repeating unit of GGGGS (SEQ ID NO:1), a heavy chain variable domain of the anti-PD-1 antibody, a second peptide linker having repeating unit of GGGGS (SEQ ID NO:1), and the polypeptide binder of TGF-β. In some embodiments, a light chain variable domain and/or a heavy chain variable domain of the anti-PD-1 antibody is derived from one or more of nivolumab, pembrolizumab, cemiplimab, dostarlimab, vopratelimab, spartalizumab, camrelizumab, sintilimab, tislelizumab, toripalimab, INCMGA00012, AMP-224, AMP-514, and acrixolimab.

Various vectors comprising one or more polynucleotides disclosed herein are also provided. Viruses for transducing or transfecting an immune cell, HSC, ESC, or pluripotent stem cell may also include a vector disclosed herein.

Additional embodiments provide methods of generating engineered T-lymphocytes (T-cells) or natural killer (NK) cells, wherein the methods include transfecting or transducing an immune cell, HSC, ESC, or pluripotent stem cell with a polynucleotide disclosed herein, and expressing the fusion protein in the immune cell, HSC, ESC, or pluripotent stem cell; and further optionally secreting a mature form of the fusion protein (i.e., cleaved from the signal peptide) from the immune cell, HSC, ESC, or pluripotent stem cell.

In some embodiments, the immune cell, HSC, ESC, or pluripotent stem cell expresses a CAR, and after the transfection/transduction, still expresses the CAR besides expressing the fusion protein comprising a polypeptide checkpoint inhibitor and a polypeptide binder of TGF-β (or a polypeptide binder of TGF-β receptor) and optionally a signal peptide at the N-terminus of the fusion protein.

In other embodiments, the immune cell, HSC, ESC, or pluripotent stem cell does not express a CAR to begin with, and after the transfection/transduction with a vector expressing the three elements a)-c) or the four elements a)-d), begins to express a CAR and the fusion protein.

In further embodiments, the method further includes detecting presence of the fusion protein in a culture medium of the transfected or transduced cells.

Additional embodiments provide methods of modifying chimeric antigen receptor (CAR)-expressing immune cells, and the methods include transfecting or transducing the CAR-expressing immune cells with a polypeptide encoding a fusion protein comprising a polypeptide checkpoint inhibitor and a polypeptide binder of transforming growth factor beta (TGF-β), so as for the CAR-expressing immune cells to express and secrete the fusion protein. In some embodiments, the fusion protein further includes a signal peptide at the N-terminus, such that the secreted protein is a mature form of the fusion protein (i.e., cleaved from the signal peptide).

Additional embodiments provide methods of treating a subject having a tumor, having undergone an anti-cancer therapy, or in need of inhibiting a tumor relapse, wherein the methods include administering to the subject a pharmaceutical composition comprising an effective amount of the genetically engineered cells disclosed herein and a pharmaceutically acceptable excipient.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1F depict generation and characterization of CAR-T cells and target cells. (1A) Schematic diagram of CAR-T cells with trap protein secretion in tumor microenvironment. (1B) Schematic representation of CAR constructs of the parental anti-CD19 CAR (CD19 CAR), anti-CD19 CAR with anti-PD-1 scFv secretion (CD19 CAR-αPD1) and anti-CD19 CAR with trap protein secretion (CD19 CAR-Trap). (1C) Expression of CARs in primary human T cells. CAR T cells were stained with biotin conjugated rat anti-mouse F(ab′)2 antibody, followed by APC-conjugated streptavidin, to detect CAR expression on the cell surface. NT indicates non-transduced T cells, which were used as a control. (1D, 1E) Expression of PD-L1 and CD19 on the cell surface of target cell lines. (1F) Secretion level of TGF-β by target cell lines. The concentrations of TGF-β in the conditioned cell culture medium were shown in bar graph (n=3, mean±SD).

FIG. 2A-2E depict characterization of trap protein. (2A and 2B) Detection of trap protein binding to 293T-PD-1 cells by anti-His-tag antibody or anti-mouse Fab antibody. Cell culture supernatant was collected from 293T cells transfected with the trap protein vector and used to incubate 293T cells with PD-1 expression at room temperature for 1 h. Cell culture soup collected from wild-type 293T cells was used as control. (2C) Trap protein purified from transfected 293T cells was analyzed by western blot. (2D and 2E) Binding ability of trap to PD-1 and TGF-β using sandwich ELISA, wherein purified trap protein was added to PD-1-Fc-coated or TGF-β-coated plates, followed by detection by anti-His-tag antibody conjugated with HRP.

FIG. 3A-3F depict the responses of CAR-T cells upon stimulation from target cells. CAR T cells were co-cultured with PC3-PDL1-CD19 cells (also termed PC3-CD19) for 16 hours in the presence of protein transport inhibitor Brefeldin A. (3A) IFN-γ, (3B) TNF-α, (3C) IL2, (3D) Ki67, (3E) granzyme B, and (3F) CD107a were measured by flow cytometry, and the percentages of marker positive cells were shown in bar graphs. NT cells were used as a control (n=3, mean±SD).

FIG. 3G and 3H depict IFN-γ expression measured by flow cytometry after CAR T cells were co-cultured with H292-CD19 cells (3G) and SKOV3-CD19 cells (3H), respectively, for 16 hours in the presence of protein transport inhibitor Brefeldin A. The percentage of IFN-γ+ T cells over total CD8+ T cells was shown in bar graphs (n=3, mean±SD; ns, not significant; **P<0.01).

FIG. 4A-4E depict in vitro functionality of CAR-T cells. (4A) Cytotoxicity of CAR-T cells against target cells. The three groups of CAR T cells were co-cultured for 24 hours with H292-CD19, SKOV3-CD19 and PC3-CD19 cells at 1:1, 3:1, and 5:1 effector-to-target ratios. NT cells were used as a control. (n=3, mean±SD). (4B) Expression of phosphorylated Smad2/3 (pSmad2/3) in T cells after incubation in different concentrations of recombinant TGF-β for 24 hours. The percentages of pSmad2/3+ T cells were shown in bar graphs. (n=3, mean±SD; ***P<0.001). (4C) Representative FACS gating for pSmad2/3+ T cells using NT cells without incubation as a negative control. (4D) Percentages of Tregs (CD4⁺CD25⁺Foxp3⁺) in different CAR-T cell groups with or without co-culture with PC3-CD19 cells for 24 hours. The data from FACS were shown in bar graphs. NT cells were used as a control. (n=3, mean±SD; ***P<0.001). (4E) CAR-T cells were co-cultured with PC3-CD19 cells for various durations. IFN-γ secretion in supernatants was quantified by ELISA. (n=3, mean±SD; ns, not significant; **P<0.01).

FIG. 5A-5F depict the expression of exhaustion markers in CAR-T cells. (5A) CD3+ T cells were shown in each panel. PD-1⁺CD8⁺ T cells were gated, and their percentage over total CD3⁺ T cells was shown in each scatterplot. (5B) The percentages of PD-1⁺CD4⁺ and PD-1⁺CD8⁺ T cells over total CD4⁺ and CD8⁺ T cells were shown in bar graphs. (n=3, mean±SD; ***P<0.001). (5C, 5D) LAG3 expression and TIM3 expression were measured by flow cytometry. The percentages of LAG3⁺CD8⁺ and TIM3⁺CD8⁺ T cells over total CD8⁺ T cells were shown in bar graphs. (n=3, mean±SD; ns, not significant, **P<0.01). (5E, 5F) PD-L1 expression was measured by flow cytometry. The percentages of PD-L1⁺CD4⁺ and PD-L1⁺CD8⁺ over total CD4⁺ and CD8⁺ T cells were shown in bar graphs. (n=3, mean±SD; *P<0.05; **P<0.01).

FIG. 5G. CAR T cells were co-cultured with H292-CD19 cells for 24 hours. (Upper left) The percentages of PD-1+CD8+ T cells over total CD8+ T cells were shown in bar graphs. (n=3, mean±SD; ***P<0.001). (Upper right) CD3+ T cells were shown in each panel. PD-1+CD8+ T cells were gated, and their percentage over total CD3+ T cells was shown in each scatterplot. CART cells were co-cultured with H292-CD19 cells for 24 hours. (Middle row) TIM3 expression and LAG3 expression were measured by flow cytometry. The percentages of TIM3+CD8+ and LAG3+CD8+ T cells over total CD8+ T cells were shown in bar graphs. (n=3, mean±SD; ns, not significant, **P<0.01). (Bottom row) PD-L1 expression was measured by flow cytometry. The percentages of PD-L1⁺CD4⁺ and PD-L1⁺CD8⁺ over total CD4⁺ and CD8⁺ T cells were shown in bar graphs. (n=3, mean±SD; *P<0.05; **P<0.01).

FIG. 511. CAR T cells were co-cultured with SKOV3-CD19 cells for 24 hours. (Upper left) The percentages of PD-1⁺CD8⁺ T cells over total CD8⁺ T cells were shown in bar graphs. (n=3, mean±SD; ***P<0.001). (Upper row) CD3⁺ T cells were shown in each panel. PD-1⁺CD8⁺ T cells were gated, and their percentage over total CD3⁺ T cells was shown in each scatterplot. CAR T cells were co-cultured with SKOV3-CD19 cells for 24 hours. (Middle row) TIM3 expression and LAG3 expression were measured by flow cytometry. The percentages of TIM3⁺CD8⁺ and LAG3⁺CD8⁺ T cells over total CD8⁺ T cells were shown in bar graphs. (n=3, mean±SD; ns, not significant, **P<0.01). (Bottom row) PD-L1 expression was measured by flow cytometry. The percentages of PD-L1⁺CD4⁺ and PD-L1+CD8⁺ over total CD4⁺ and CD8⁺ T cells were shown in bar graphs. (n=3, mean±SD; *P<0.05; **P<0.01).

FIGS. 6A-61I depict the antitumor efficacy, infiltration and functionality of CAR-T cells in vivo. (6A) Schematic representation of the in vivo experimental procedure. NSG mice were subcutaneously (s.c.) injected with 3×10⁶ of PC3-CD19 tumor cells into the right flank. After 16 days, when the tumors grew to 75-100 mm³, 2×10⁶ of CD19 CAR, CD19 CAR-αPD-1, or CAR19 CAR-Trap T cells were adoptively transferred through intravenous (i.v.) injection. Tumor volume was measured every other day. The mice were euthanized for analysis on day 12 post-treatment. (6B) Tumor growth curve for mice treated with non-transduced (NT), CD19 CAR, CD19 CAR-αPD-1, or CAR19 CAR-Trap T cells. (n=5, mean±SD; **P<0.01; ***P<0.001). (6C) The ratio of CD8⁺ versus CD4⁺ T cells in the tumor. (n=5, mean±SD; ***P<0.001). (6D) The percentage of PD-1⁺ TILs over total TILs. (n=5, mean±SD; *P<0.05). (6E) The percentage of Tregs (CD45⁺CD4⁺CD25⁺Foxp3⁺ cells) over total TILs. (n=5, mean±SD; **P<0.01; ***P<0.001). (6F) The percentage of pSmad2/3⁺ TILs over total TILs. (n=5, mean±SD; **P<0.01). (6G) The percentage of CD45⁺ T cells in the tumor, blood, spleen and bone marrow tissues of PC3-CD19 tumor-bearing mice of different groups. (n=5, mean±SD; *P<0.05; **P<0.01; ***P<0.001). (6H) A representative FACS scatter plot of the percentage of CD45⁺ T cells in the tumor, blood, spleen and bone marrow tissues of different groups.

FIG. 61 depicts tumor samples collected from CD19 CAR, CD19 CAR-αPD1 or CD19 CAR-Trap T cell treated groups on day 12 post-treatment were analyzed for memory status. Samples were stained for human CD45RO and CD62L, and measured by flow cytometry. The average percentages of naïve T cells, effector memory T cells, central memory T cells and effector T cells were shown in pie graphs.

FIGS. 7A-7E depict the long-term antitumor efficacy and immune surveillance of CAR-T cells in vivo. (7A) Schematic representation of the in vivo experimental procedure. NSG mice were s.c. injected with 3×10⁶ of PC3-CD19 tumor cells into the right flank. After 16 days, when the tumors grew to ˜100 mm³, 4×10⁶ of CD19 CAR, CD19 CAR-αPD-1, or CAR19 CAR-Trap T cells were adoptively transferred through i.v. injection. Tumor volume was measured every other day. The mice were monitored until they met the endpoint. On day 60 post-treatment, the mice in CD19 CAR-Trap group were rechallenged with 1×10⁶ PC3-PDL1-CD19 tumor cells, and were monitored for another 2 weeks. (7B) Tumor growth curve for mice treated with non-transduced (NT), CD19 CAR, CD19 CAR-αPD-1, or CD19 CAR-Trap T cells. (n=8, mean±SD; ***P<0.001). (7C) Waterfall plot analysis of tumor reduction on day 12 post treatment for various treatment groups. (7D) Body weight curve for mice in various treatment groups. (n=8, mean±SD). (7E) Survival of PC3-CD19 tumor-bearing NSG mice after indicated treatment. Overall survival curves were plotted using the Kaplan-Meier method.

FIG. 7F. Eight mice of CD19 CAR-Trap group were bled on day 61 post-treatment of 4×10⁶ CAR-T cells. The blood sample were lysed to remove red blood cells and then processed for T cell analysis. The percentages of T cells in blood of each mouse were shown in bar graphs.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs.

“Chimeric antigen receptor” or “CAR” or “CARs” as used herein refers to engineered receptors, which graft an antigen specificity onto cells (for example T cells such as naïve T cells, central memory T cells, effector memory T cells or combination thereof). CARs are also known as artificial or engineered T-cell receptors, chimeric T-cell receptors or chimeric immunoreceptors. In various embodiments, CARs are recombinant polypeptides comprising an antigen-specific domain (ASD), a hinge region (HR), a transmembrane domain (TMD), co-stimulatory domain (CSD) and an intracellular signaling domain (ISD). In some embodiments, a CAR comprises (a) a zeta chain portion comprising the intracellular domain of human CD3 chain, (b) a costimulatory domain (CSD), which is intracellular domain of a costimulatory molecule, and (c) an antigen-specific domain (ASD), which specifically interacts with an antigen.

“Antigen-specific domain” (ASD) or “antigen-specific targeting domain” refers to the portion of the CAR that specifically binds the antigen on the target cell. In some embodiments, the ASD of the CARs comprises an antibody or a functional equivalent thereof or a fragment thereof or a derivative thereof. The targeting regions may comprise full length heavy chain, Fab fragments, single chain Fv (scFv) fragments, divalent single chain antibodies or diabodies, each of which are specific to the target antigen. In some embodiments, almost any molecule that binds a given antigen with high affinity can be used as an ASD, as will be appreciated by those of skill in the art. In some embodiments, the ASD comprises T cell receptors (TCRs) or portions thereof.

“Hinge region” (HR) as used herein refers to the hydrophilic region which is between the ASD and the TMD. The hinge regions include but are not limited to Fc fragments of antibodies or fragments or derivatives thereof, hinge regions of antibodies or fragments or derivatives thereof, CH2 regions of antibodies, CH3 regions of antibodies, artificial spacer sequences or combinations thereof. Examples of hinge regions include but are not limited to CD8a hinge, and artificial spacers made of polypeptides which may be as small as, for example, Gly3 or CH1 and CH3 domains of IgGs (such as human IgG4). In some embodiments, the hinge region is any one or more of (i) a hinge, CH2 and CH3 regions of IgG4, (ii) a hinge region of IgG4, (iii) a hinge and CH2 of IgG4, (iv) a hinge region of CD8a, (v) a hinge, CH2 and CH3 regions of IgG1, (vi) a hinge region of IgG1 or (vi) a hinge and CH2 region of IgG1. Other hinge regions will be apparent to those of skill in the art and may be used in connection with alternate embodiments of the invention.

“Transmembrane domain” (TMD) as used herein refers to the region of the CAR which crosses the plasma membrane. The transmembrane domain of the CAR of the invention is the transmembrane region of a transmembrane protein (for example Type I transmembrane proteins), an artificial hydrophobic sequence or a combination thereof. Other transmembrane domains will be apparent to those of skill in the art and may be used in connection with alternate embodiments of the invention. In some embodiments, the TMD of the CAR comprises a transmembrane domain selected from the transmembrane domain of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD1 1a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R a, ITGA1, VLA1, CD49a, ITGA4, IA4, CD49D, ITGA6, VLA-6, CD49f, ITGAD, CDl ld, ITGAE, CD103, ITGAL, CDl la, LFA-1, ITGAM, CD1 lb, ITGAX, CDl lc, ITGB1, CD29, ITGB2, CD18, LFA-1, ITGB7, TNFR2, DNAM1(CD226), SLAMF4 (CD244, 2B4), CD84, CD96 (Tactile), CEACAM1, CRT AM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C.

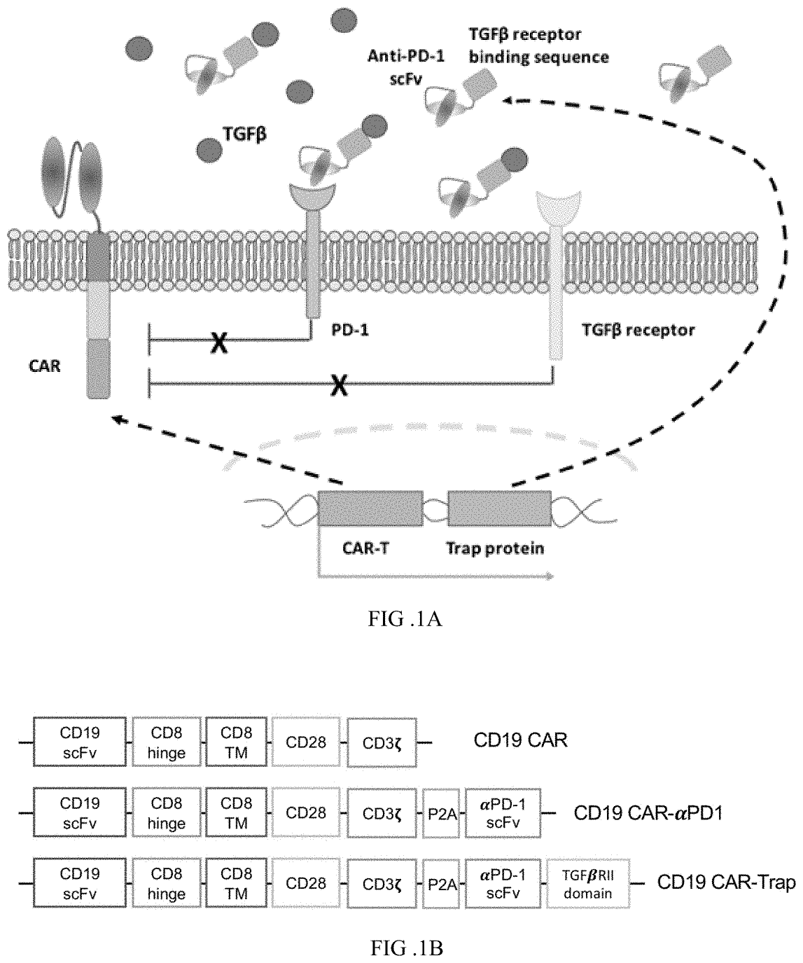

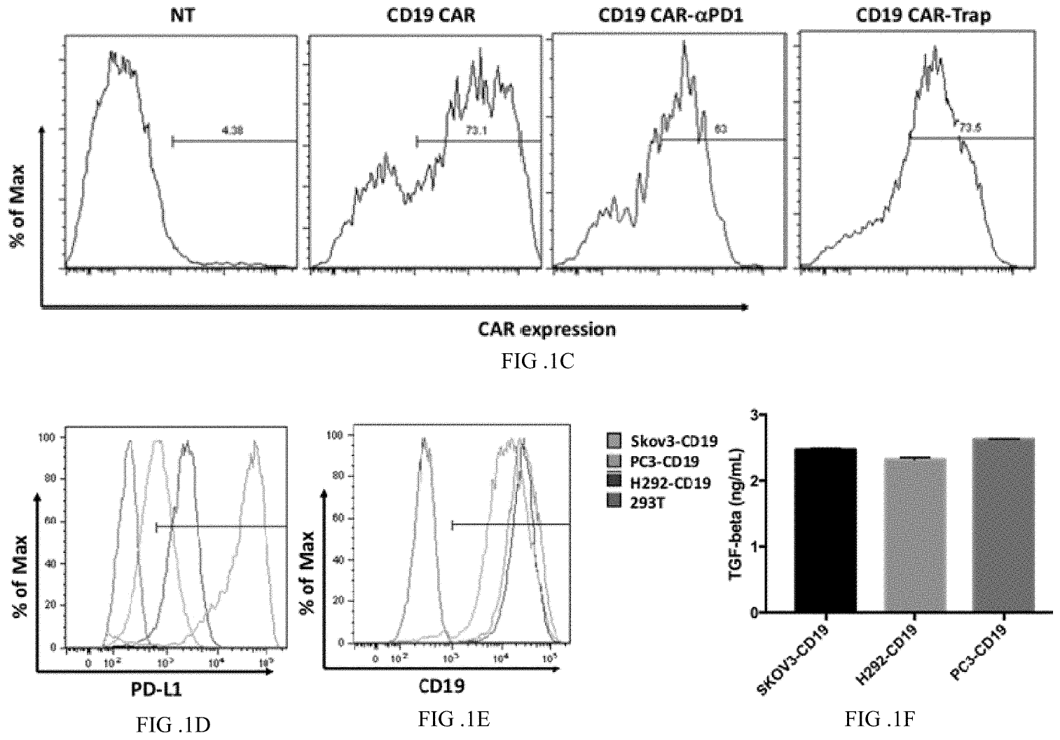

“Co-stimulatory domain” (CSD) as used herein refers to the portion of the CAR which enhances the proliferation, survival and/or development of memory cells. In various embodiments, a CSD comprises the intracellular domain of a costimulatory molecule, and costimulatory molecules are cell surface molecules other than antigen receptors or their ligands that are required for an efficient response of lymphocytes to antigen. The CARs of the invention may comprise one or more co-stimulatory domains. Examples of costimulatory molecules include members of the TNFR superfamily, or CD28, CD137 (4-1BB), CD134 (OX40), DAP10, ICOS, CD27, CD2, CD5, ICAM-1, LFA-1(CD11a/CD18), Lck, TNFR-I, TNFR-II, Fas, CD30, CD40 or combinations thereof. Other co-stimulatory domains (e.g., from other proteins) will be apparent to those of skill in the art and may be used in connection with alternate embodiments of the invention. Thus, while the invention is exemplified in FIG. 1B primarily with CD28 as the co-stimulatory domain, other costimulatory elements are within the scope of the invention. For example, a CAR containing the intracellular domain of 4-1BB, ICOS, or DAP-10 are also suitably employed in the invention.

“Intracellular signaling domain” (ISD) or “cytoplasmic domain” as used herein refers to the portion of the CAR which transduces the effector function signal and directs the cell to perform its specialized function. Examples of domains that transduce the effector function signal include but are not limited to the z chain of the T-cell receptor complex or any of its homologs (e.g., h chain, FceR1g and b chains, MB1 (Iga) chain, B29 (Igb) chain, etc.), human CD3 zeta chain, CD3 polypeptides (D, d and e), syk family tyrosine kinases (Syk, ZAP 70, etc.), src family tyrosine kinases (Lck, Fyn, Lyn, etc.) and other molecules involved in T-cell transduction, such as CD2, CD5 and CD28. Other intracellular signaling domains will be apparent to those of skill in the art and may be used in connection with alternate embodiments of the invention.

“Peptide linker” (L) or “linker domain” or “linker region” as used herein refer to an oligo- or polypeptide region from about 1 to 100 amino acids in length, which links together any of the domains/regions of the CAR, checkpoint inhibitor, and/or binder of TGFβ or receptor of TGFβ.

In some embodiments, a peptide linker is composed of flexible residues like small, non-polar (e.g., glycine) and polar (e.g., serine or threonine), so that the adjacent protein domains are free to move relative to one another. The small size of these amino acids provides flexibility and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. For example, polypeptide sequence having a repeating unit of GGGGS (SEQ ID NO:1), repeated for the copy number, n=2, 3, 4, 5, 6, 7, 8, 9, or 10, or more. By adjusting the copy number “n”, the length of this GS linker can be optimized to achieve appropriate separation of the functional domains, or to maintain necessary inter-domain interactions. Longer linkers may be used when it is desirable to ensure that two adjacent domains do not sterically interfere with one another. Other suitable flexible linkers may be also rich in small or polar amino acids such as Gly and Ser, but can contain additional amino acids such as Thr and Ala to maintain flexibility, as well as polar amino acids such as Lys and Glu to improve solubility. For example, KESGSVSSEQLAQFRSLD (SEQ ID NO:2) and EGKSSGSGSESKST (SEQ ID NO:3) are flexible linkers, suitable for the construction of a bioactive scFv. The Gly and Ser residues in the linker were designed to provide flexibility, whereas Glu and Lys were added to improve the solubility.

Other embodiments provide that peptide linkers are cleavable. Examples of cleavable linkers include 2A linkers (for example T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. In other embodiments, the linker sequences may comprise Asp-Val/Ile-Glu-X-Asn-Pro-Gly^{(2A)}-Pro^{(2B)} (SEQ ID NO:21), a consensus octamer motif of 2A peptide, which results in cleavage between the 2A glycine and the 2B proline. Other cleavable linkers include protease-sensitive peptide linkers, such that the peptide linkers are cleaved in a protease-rich environment, such as in a tumor microenvironment. Additional linkers are described in Adv Drug Deliv Rev. 2013 Oct. 15; 65(10): 1357-1369, and may be used in connection with alternate embodiments of the invention.

The term “antibody” refers to an immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies, single domain antibodies, chimeric antibodies, diabodies and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g. papain cleavage) or via chemical cleavage of intact antibodies.

The term “antibody fragment” refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., PNAS USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

“Single chain variable fragment”, “single-chain antibody variable fragments” or “scFv” antibodies as used herein refers to forms of antibodies comprising the variable regions of only the heavy (V_{H}) and light (V_{L}) chains, connected by a linker peptide. The scFvs are capable of being expressed as a single chain polypeptide. The scFvs retain the specificity of the intact antibody from which it is derived. The light and heavy chains may be in any order, for example, V_{H}-linker-V_{L} or V_{L}-linker-V_{H}, so long as the specificity of the scFv to the target antigen is retained.

The terms “T-cell” and “T-lymphocyte” are interchangeable and used synonymously herein. Examples include but are not limited to naïve T cells, central memory T cells, effector memory T cells or combinations thereof.

The term “secretion,” “secretable,” or its grammatical variants as used herein refers to protein secretion. Without wishing to be bound by a theory, protein secretion is believed to be a multistep process that involves vesicle biogenesis, cargo loading, concentration and processing, vesicle transport and targeting, vesicle docking and Ca2+-dependent vesicular fusion with the plasma membrane.

The term “signal peptide”, or “leading sequence,” refers to a polypeptide when expressed with, or fused to, a nascent protein in a cell, augments secretion of the nascent protein from the cell. Signal peptides, located at the N-terminus (upstream) of nascent secreted proteins, generally characteristically have three domains: (1) a basic (positively charged) domain at the N-terminus, (2) a central hydrophobic core, and (3) a carboxy-terminal cleavage region. Signal peptides are also generally 20-40 amino acid residues in length. Without wishing to be bound by a particular theory, the basic N-terminus and the hydrophobic core allows for the signal peptide to anchor firmly onto the membrane of endoplasmic reticulum, thereby facilitating protein translocation in the cell.

Preferably a signal peptide is a mammalian signal peptide. An example of a signal peptide is IL-2 signal peptide or IL-2 leading sequence, preferably human IL-2 leading sequence. A variant of IL-2 signal peptide may have alterations in the basic and/or hydrophobic domains of the IL-2 signal peptide. Zhang et al. describes some alterations in the IL-2 signal peptide augment secretion of proteins compared to wild-type IL-2 signal peptide. See Zhang et al., J Gene Med 2005; 7: 354-365. Variants of a signal peptide may typically result in a greater secretion of nascent protein when the basicity, the hydrophobicity, or both of the signal peptide are increased compared to the wild type version. Hydrophobicity and basicity of amino acids, especially the index giving relative hydrophobicity or relative basicity among all amino acids, are known in the art, e.g., in amino acids reference chart provided in www.sigmaaldrich.com/US/en/technical-documents/technical-article/protein-biology/protein-structural-analysis/amino-acid-reference-chart.

As used herein, the term “vector” is understood to mean any nucleic acid comprising a nucleotide sequence competent to be incorporated into a host cell and to be recombined with and integrated into the host cell genome, or to replicate autonomously as an episome. Such vectors include linear nucleic acids, plasmids, phagemids, cosmids, RNA vectors, viral vectors and the like. Non-limiting examples of a viral vector include a retrovirus, an adenovirus and an adeno-associated virus. In some embodiments, the vectors are mammalian expression plasmids.

The term “cancer,” “neoplasm” or “tumor,” including grammatical variations thereof, refers to new and abnormal growth of tissue, which may be benign or cancerous. In a related aspect, the neoplasm is indicative of a neoplastic disease or disorder, including but not limited, to various cancers. For example, such cancers can include prostate, pancreatic, biliary, colon, rectal, liver, kidney, lung, testicular, breast, ovarian, pancreatic, brain, and head and neck cancers, melanoma, sarcoma, multiple myeloma, leukemia, lymphoma, and the like.

The term “subject” or “patient” refers to a human or vertebrate animal including a dog, cat, horse, cow, pig, sheep, goat, chicken, monkey, rat, and mouse. In some embodiments, the subject is a human with a cancer, such as prostate cancer, ovarian cancer, or breast cancer, where active TGF-β signaling is prevalent in the cancer environment.

As used herein the term “about” or “approximately” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

We have developed new CAR-T cells with the capacity to secret (release) trap proteins (FIG. 1A) consisting of, consisting essentially of, or comprising an anti-PD-1 scFv and a TGFβRII ectodomain sequence. It is conceived to enhance CAR-T therapy for tumors such as solid tumors. In a xenograft mouse model, CAR T cells with trap protein secretion exhibited enhanced tumor infiltration, expansion, antitumor efficacy compared with the parental CAR-T cells and anti-PD-1 self-secreting CAR-T cells. We demonstrated that trap protein secretion empowers CAR-T cells with a great potential to eradicate solid tumors and to prevent tumors relapse.

The immunosuppression from TGF-β is potent and associated with immune checkpoint signaling pathways. We previously developed CAR-T cells secreting checkpoint inhibitors to block the PD-1/PD-L1 interaction. Compared with systemic administration of PD-1 antibody with CAR-T cells, anti-PD-1 self-secreting CAR-T cells have proven more functional and expandable, and more efficient at mediating tumor eradication. Studies have found that active TGF-β signaling in TME might be responsible for the poor response rates observed in clinical trials of checkpoint inhibitors, especially in the treatment for prostate cancer, ovarian cancer and breast cancer. Ravi et al. reported a bifunctional antibody-ligand trap protein comprising an anti-PD-L1 antibody fused with a TGFβRII ectodomain sequence. Through the dual-targeting effect, the trap protein simultaneously blocks immune checkpoints and inhibits TGF-β-mediated differentiation of Tregs, thereby indicating a promising strategy for cancers that fail to respond to immune checkpoint inhibitors. Our strategy herein to overcome the current hurdles faced by CAR-T therapy in solid tumor treatment, or in other tumor treatment, involves the engineering of CAR-T cells to secret bifunctional trap proteins into the tumor microenvironment, simultaneously targeting checkpoint molecule (e.g., PD-1, PD-L1, CTLA-4) and soluble immunosuppressive molecule (e.g., TGF-β). The in vitro studies have shown that the secreted trap protein significantly enhanced CAR-T cell proliferation and production of cytotoxicity-related molecules upon antigen stimulation, whereas inhibiting the activation of Tregs and upregulation of immune-checkpoint molecules in vitro. In the xenograft mouse model, we established that CAR-T cells with trap protein secretion exhibited not only superior antitumor efficacy but also enhanced tumor infiltration and expansion, when compared with conventional CAR-T cells and CAR-T cells with anti-PD-1 scFv secretion.

This proof-of-concept study provides support for the strategy of utilizing self-secreting CAR-T cells for co-targeting two immunosuppressive mechanisms in the TME. This three-in-one approach could potentially improve the clinical outcome of CAR-T therapy by rescuing CAR-T cells from immunosuppression and enhancing the expansion and functionalities of CAR-T cells. Moreover, the protein drug secreted by CAR-T cells could achieve a localized accumulation at tumor site, which may improve the safety profile of combinational CAR-T therapy by avoiding toxicities associated with systemic administration of protein drugs.

Various embodiments provide polynucleotides encoding a fusion protein, wherein the fusion protein comprises a polypeptide checkpoint inhibitor and a polypeptide binder of transforming growth factor beta (TGF-β), optionally operably linked by a peptide linker. Other embodiments provide polynucleotides encoding a fusion protein, wherein the fusion protein comprises a polypeptide checkpoint inhibitor and a polypeptide binder of transforming growth factor beta receptor (TGF-βR), optionally operably linked by a peptide linker. Further embodiments provide that the polynucleotide further encodes a CAR, wherein the CAR and the fusion protein is operably linked by a cleavable peptide linker, e.g., a self-cleaving peptide or a protease-cleavable peptide.

In some embodiments, the polypeptide checkpoint inhibitor comprises an antigen-binding fragment of one or more of an anti-programmed cell death protein 1 (PD-1) antibody, an anti-lymphocyte-activation gene 3 (LAG3) antibody, an anti-T cell immunoglobulin domain and mucin domain-containing protein 3 (TIM3) antibody, and an anti-T-lymphocyte antigen-4 (CTLA-4) antibody.

In some embodiments, the polypeptide binder of TGF-β comprises a TGF-β-binding fragment of a TGF-β receptor or a TGF-β-binding fragment of an anti-TGF-β antibody. In some embodiments, the polypeptide binder of TGF-βR comprises TGF-β or a fragment thereof. In some embodiments, the polypeptide that binds transforming growth factor beta (TGF-β) disables autocrine/paracrine TGF-β in the target cell microenvironment.

Various embodiments provide genetically engineered cells, wherein a genetically engineered cell comprises one or more polynucleotides encoding:
a chimeric antigen receptor (CAR), and
a fusion protein comprising:
   a polypeptide that binds an immune checkpoint protein or a polypeptide that binds a ligand of the immune checkpoint protein, and
   a polypeptide that binds transforming growth factor beta (TGF-β) or a polypeptide that binds a TGF-β receptor.

In some embodiments, the fusion protein further comprises a signal peptide at the N-terminus; hence upon expression, a mature form of the fusion protein—cleaved from the signal peptide—is secreted by the genetically engineered cells. In some embodiments, a signal peptide comprises a basic domain at the N-terminus, a central hydrophobic core, and a carboxy-terminal cleavage region (also called a lytic region).

In some embodiments, a genetically engineered cell is provided, which expresses a (i) CAR, (ii) a checkpoint inhibitor which is a polypeptide, and (iii) a polypeptide specifically binding TGF-β or TGF-β receptor, thereby inhibiting TGF-β/TGF-β receptor interaction. In further embodiments, the genetically engineered cell further express (iv) a signal peptide at the N-terminus of a fusion protein comprising the signal peptide, the polypeptide checkpoint inhibitor, and a polypeptide binder of TGF-β or TGF-β receptor.

In further embodiments, the genetically engineered cell expresses the CAR and secretes the fusion protein. In various aspects, the fusion protein co-targets PD-1 and TGF-β, or co-targets PD-L1 and TGF-β, or co-targets PD-1 and TGF-β receptor, or co-targets PD-L1 and TGF-β receptor, or co-targets CTLA-4 and TGF-β, or co-targets CTLA-4 and TGF-β receptor.

Further embodiments of CAR-T therapy are conceived. In some embodiments, the genetically engineered cells are T-cells, which contain a CAR with an antigen-specific targeting region which targets solid tumor antigens, such as prostate-specific membrane antigen (PSMA) or human epidermal growth factor receptor 2 (HER2). In other embodiments, the genetically engineered T-cells contain a CAR with an antigen-specific targeting region which targets a B-cell-related disorder. In some embodiments, the simultaneous inhibition of PD-1/PD-L1 and TGF-signaling pathway by the trap protein secreted from CAR-T cells is assayed for the antitumor potential in syngeneic mouse models. Since this self-secretion platform is modular and flexible, it is conceived that a CAR-expressing T-cell can have combinations of different antibodies and binding sequences for receptors or ligands. For example, it is a combination of anti-PD-L1 antibody or anti-cytotoxic T-lymphocyte associated protein 4 (CTLA-4) antibody with TGF-β.

In some embodiments, the polypeptide that targets CTLA-4 is derived from CTLA-4 blocking antibody (Ipilimumab), e.g., a scFv, or V_{H}, or V_{L}, or comprising both V_{H} and V_{L}, of ipilimumab. In some aspects, it is the light chain variable region, the heavy chain variable region, or both of the antibody that are used as the polypeptide that targets a protein of interest.

In some embodiments, the polypeptide that targets PD-1 is derived from an anti-PD-1 antibody, such as Pembrolizumab, Nivolumab, Cemiplimab, and/or Dostarlimab, e.g., a scFv, or V_{H}, or V_{L}, or comprising both V_{H} and V_{L}, of Pembrolizumab, Nivolumab, Cemiplimab, and/or Dostarlimab. In some aspects, it is the light chain variable region, the heavy chain variable region, or both of the antibody that are used as the polypeptide that targets a protein of interest. Additional anti-PD-1 antibodies are suitable for deriving the sequence of a polypeptide that binds to PD-1, and additional anti-PD-1 antibodies include but are not limited to Vopratelimab, Spartalizumab, Camrelizumab, Sintilimab, Tislelizumab, Toripalimab, INCMGA00012, AMP-224, AMP-514, and Acrixolimab. Additional anti-PD-L1 antibodies are suitable for deriving the sequence of a polypeptide that binds to PD-L1, and additional anti-PD-L1 antibodies include but are not limited to KN035, Cosibelimab, AUNP12, CA-170, and BMS-986189.

In some embodiments, the polypeptide that targets PD-L1 is derived from an anti-PD-L1 antibody, such as Atezolizumab, Avelumab, and/or Durvalumab, e.g., a scFv, or V_{H}, or V_{L}, or comprising both V_{H} and V_{L}, of Atezolizumab, Avelumab, and/or Durvalumab. In some aspects, it is the light chain variable region, the heavy chain variable region, or both of the antibody that are used as the polypeptide that targets a protein of interest.

In some embodiments, the fusion protein comprises an antibody or antibody fragment targeting PD-1, PD-L1 or CTLA-4, which is fused to a TGFβRII ectodomain sequence (forming anti-PD-1-TGFβRII, anti-PD-L1-TGFβRII, or anti-CTLA-4-TGFβRII, respectively). In further embodiments, the fusion protein is a single monomeric polypeptide comprising (1) a VH targeting PD-1, PD-L1 or CTLA4, (2) a VL targeting PD-1, PD-L1 or CTLA4, and (3) TGFβ receptor ectodomain. In other embodiments, the fusion protein is a single monomeric polypeptide comprising (1) a VH targeting PD-1, PD-L1 or CTLA4 and (3) TGFβ receptor ectodomain. In yet other embodiments, the fusion protein is a single monomeric polypeptide comprising (2) a VL targeting PD-1, PD-L1 or CTLA4 and (3) TGFβ receptor ectodomain. The fusion protein aims to sequester and disable autocrine/paracrine TGFβ in the target cell microenvironment which blocking immune checkpoint protein interactions. Preferably, the fusion protein simultaneously disables immune checkpoints and counteracts TGFβ-mediated differentiation of Tregs and immune tolerance, thereby providing a more effective immunotherapeutic strategy against cancers especially those that fail to respond to current immune checkpoint inhibitors.

In various aspects, the C terminus of an anti-PD-1, anti-PD-L1 or anti-CTLA-4 antibody or its monomeric fragment (e.g., VH, VL, or scFv) is fused with a ligand-binding sequence of the extracellular domain of TGFβRII via a flexible linker peptide, such as (GGGGS (SEQ ID NO:1))ₙ, wherein n=3, 2, 4, 5, or another integer between 1 and 10.

In some embodiments, a polypeptide that binds TGFβ comprises an extracellular domain or ligand-binding sequence of one of the following receptors: Transforming growth factor-beta receptor (TGF-βRII, TGF-βRIIb, or TGF-βRIII). In some embodiments, the ligand-binding sequence of TGFβ receptor ectodomains is described in U.S. Pat. No. 8,993,524, which is incorporated by reference in its entirety. For example, the TGFβ-binding sequence of TGFβRII extracellular domain has an amino acid sequence of:

(SEQ ID NO: 4)
TIPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKCI
MKEKKKPGETFFMCSCSSDECNDNIIFSEEYNTSNPD.

In other embodiments, a polypeptide that binds TGFβ receptor comprises TGFβ or its active ligand fragment.

In some embodiments, the polypeptide that binds TGFβ or the polypeptide that binds TGFβ receptor is fused (or operably linked) to the C-terminus of a polypeptide that binds an immune checkpoint protein or a polypeptide that binds a ligand of the immune checkpoint protein, forming a fusion protein. In some embodiments, the polypeptide that binds TGFβ or the polypeptide that binds TGFβ receptor is fused (or operably linked) to the N-terminus of a polypeptide that binds an immune checkpoint protein or a polypeptide that binds a ligand of the immune checkpoint protein, forming a fusion protein. In further embodiments, the polypeptide that binds TGFβ or the polypeptide that binds TGFβ receptor is fused (or operably linked) to the polypeptide that binds an immune checkpoint protein or the polypeptide that binds a ligand of the immune checkpoint protein via a linker that is not a protease-labile or self-cleaving linker, forming a fusion protein.

In some embodiments, a polynucleotide or a vector or retrovirus comprising the polynucleotide is provided, which contains a 2A peptide coding sequence operably linked to a CAR and the fusion protein disclosed herein. In some embodiments, the 2A peptide coding sequence is operably linked on the 5′ terminus to the 3′ terminus of the CAR, and the 2A peptide coding sequence is operably linked on the 3′ terminus to the 5′ terminus of the fusion protein. In other embodiments, the 2A peptide coding sequence is operably linked on the 3′ terminus to the 5′ terminus of the CAR, and the 2A peptide coding sequence is operably linked on the 5′ terminus to the 3′ terminus of the fusion protein.

2A peptides, also referred to as 2A self-cleaving peptides, is generally a class of 18-22 amino acid-long peptides, which can induce ribosomal skipping during translation, thereby generating polyproteins by causing the ribosome to fail at making a peptide bond. Without wishing to be bound by a theory, the cleavage is triggered by ribosomal skipping of the peptide bond between the proline (P) and glycine (G) in C-terminal of a 2A peptide, resulting in the peptide located upstream of the 2A peptide to have extra amino acids on its C-terminal end while the peptide located downstream the 2A peptide will have an extra proline on its N-terminal end.

In some embodiments, a 2A peptide encoded by the polynucleotide disclosed herein is a T2A peptide, which is derived from thosea asigna virus 2A. For example, a T2A peptide comprises an amino acid sequence of EGRGSLLTCGDVEENPGP (SEQ ID NO:5). An optional linker “GSG” (Gly-Ser-Gly) may be added on the N-terminal of the T2A peptide, which may improve efficiency.

In some embodiments, a 2A peptide encoded by the polynucleotide disclosed herein is a P2A peptide, which is derived from porcine teschovirus-12A. For example, a P2A peptide has an amino acid sequence of ATNFSLLKQAGDVEENPGP (SEQ ID NO:6). An optional linker “GSG” (Gly-Ser-Gly) may be added on the N-terminal of the P2A peptide, which may improve efficiency.

In some embodiments, a 2A peptide encoded by the polynucleotide disclosed herein is an E2A peptide, which is derived from equine rhinitis A virus. For example, an E2A peptide has an amino acid sequence of QCTNYALLKLAGDVESNPGP (SEQ ID NO:7). An optional linker “GSG” (Gly-Ser-Gly) may be added on the N-terminal of the E2A peptide, which may improve efficiency.

In some embodiments, a 2A peptide encoded by the polynucleotide disclosed herein is an F2A peptide, which is derived from foot-and-mouth disease virus 18. For example, an F2A peptide has an amino acid sequence of VKQTLNFDLLKLAGDVESNPGP (SEQ ID NO:8). An optional linker “GSG” (Gly-Ser-Gly) may be added on the N-terminal of the F2A peptide, which may improve efficiency.

In some embodiments, a nucleic acid polymer or a vector or retrovirus comprising the polynucleotide is provided, which contains a 2A peptide coding sequence operably linked to a CAR and a fusion protein, wherein the fusion protein comprises a signal peptide, a polypeptide checkpoint inhibitor, and a polypeptide binder of TGF-β, wherein the signal peptide is at the N-terminus of the fusion protein. In some embodiments, In various implementations, upon expression of the polynucleotide, vector, or retrovirus in a cell, the CAR is cleaved from the fusion, and the fusion protein upon secretion from the cell is cleaved from the signal peptide, hence resulting in a mature fusion protein (comprising the polypeptide checkpoint inhibitor and the polypeptide binder of TGF-β, but lacking the signal peptide) secreted from the cell.

Exemplary signal peptides include those naturally occurring signal peptides and variants thereof resulting from mutations of the naturally occurring ones. For example, naturally occurring signal peptides include those associated with IL-2 precursor, somatostatin, urocortin precursor, reticulocalbin 1 precursor, anticoagulant protein C, lysozyme C precursor, fibrillin1 precursor, microsomal protein, endoplasmic reticulum protein 29 precursor, apolipoprotein C-II precursor, endoplasmin precursor, immunoglobulin heavy chain binding protein, GM-CSF, galanin precursor, IL-11, IL-9 receptor, chondroadherin precursor, complement 6 precursor, insulin receptor, native tuna GH, human GABA-A receptor, or IgG light chain. The sequence of each of these naturally occurring signal peptides is described in Zhang et al., J Gene Med 2005; 7: 354-365, which is incorporated by reference herein. For example, the signal peptide associated with IL-2 precursor, also referred to as “human IL-2 leading sequence” in the Examples, has an amino acid sequence of: MYRMQLLSCIALSLALVTNS (SEQ ID NO:22), wherein the first three amino acid residues MYR is the basic domain at the N-terminus, followed by the next 9 contiguous amino acid residues which is the hydrophobic domain, and the last 8 contiguous amino acid residues which is the lytic domain.

In some embodiments, variants (especially functional variants) of naturally occurring signal peptides are included in the polypeptide encoding the fusion protein; that is, a polypeptide from 5′ to 3′ encodes at least: a signal peptide or its functional variant, a polypeptide checkpoint inhibitor, and a polypeptide binder of TGF-β or TGF-β receptor. Variants of a naturally occurring (or wild type) signal peptide may be those having one or more mutations, insertions or deletions at one or more amino acid residues. Functional variants of a naturally occurring signal peptide include variants that result in at least 50%, 60%, 70%, 80%, 90%, 100% or more of the secretion rates or secretion amount of a nascent protein compared to the naturally occurring signal peptide. In some embodiments, variants having an increased basicity, an increased hydrophobicity, or both compared to the naturally occurring counterpart are functional variants of the naturally occurring signal peptide. In some embodiments, variants having an increased basicity, an increased hydrophobicity, or both compared to the naturally occurring counterpart leads to more secretion rates or secretion amounts compared to the naturally occurring counterpart.

Variants with an increased basicity include those having a replaced amino acid residue in the basic domain changing from a non-basic amino acid to a basic amino acid, and/or those having one or more basic amino acids inserted in the basic domain of the naturally occurring counterpart. Basic amino acids include Arg, His, and Lys. In some embodiments, Arg, Lys, or both are inserted in the basic domain of a naturally occurring signal peptide, thereby forming a functional variant with improved secretion ability over the naturally occurring version.

Variants with an increased hydrophobicity include those having a replace amino acid residue in the hydrophobic core of a naturally occurring signal peptide changing from a relative less hydrophobic amino acid into a relatively more hydrophobic amino acid, and/or those having an inserted amino acid with a high hydrophobicity index. The hydrophobicity index is a measure of the relative hydrophobicity, wherein glycine is given 0 value (considered neutral) and other amino acids are normalized to glycine. For example, Phe, Ile, Trp, Leu, Val, and Met (in generally descending order) are very hydrophobic at pH7; and Leu, Ile, Phe, Trp, Val, and Met (in generally descending order) are very hydrophobic at pH2. In some embodiments, “neutral” amino acids such as Ser or Gly are replaced with Ile in the hydrophobic core of a naturally occurring signal peptide, thereby forming a functional variant of the naturally occurring signal peptide.

In some embodiments, the CAR comprises: an antigen-specific targeting region, an extracellular spacer domain, a transmembrane domain, a co-stimulatory domain, and an intracellular signaling domain.

In some embodiments, the antigen-specific targeting region binds an antigen specific for cancer, inflammatory disease, neuronal disorder, diabetes, cardiovascular disease, infectious disease, or B-cell associated disease. Examples of antibodies for deriving scFv, VH, VL and antigen-binding fragments and incorporation as or into the antigen-specific targeting regions of CARs include, but are not limited, to antibodies such as trastuzumab (anti-HER2/neu antibody); Pertuzumab (anti-HER2 mAb); cetuximab (chimeric monoclonal antibody to epidermal growth factor receptor, EGFR); panitumumab (anti-EGFR antibody); nimotuzumab (anti-EGFR antibody); Zalutumumab (anti-EGFR mAb); Necitumumab (anti-EGFR mAb); MDX-210 (humanized anti-HER-2 bispecific antibody); MDX-210 (humanized anti-HER-2 bispecific antibody); MDX-447 (humanized anti-EGF receptor bispecific antibody); Rituximab (chimeric murine/human anti-CD20 mAb); Obinutuzumab (anti-CD20 mAb); Ofatumumab (anti-CD20 mAb); Tositumumab-1131 (anti-CD20 mAb); Ibritumomab tiuxetan (anti-CD20 mAb); Bevacizumab (anti-VEGF mAb); Ramucirumab (anti-VEGFR2 mAb); Ranibizumab (anti-VEGF mAb); Aflibercept (extracellular domains of VEGFR1 and VEGFR2 fused to IgG1 Fc); AMG386 (angiopoietin-1 and -2 binding peptide fused to IgG1 Fc); Dalotuzumab (anti-IGF-1R mAb); Gemtuzumab ozogamicin (anti-CD33 mAb); Alemtuzumab (anti-Campath-1/CD52 mAb); Brentuximab vedotin (anti-CD30 mAb); Catumaxomab (bispecific mAb that targets epithelial cell adhesion molecule and CD3); Naptumomab (anti-5T4 mAb); Girentuximab (anti-Carbonic anhydrase ix); or Farletuzumab (anti-folate receptor). Other examples include antibodies such as PANOREX™ (17-1A) (murine monoclonal antibody); BEC2 (anti-idiotypic mAb, mimics the GD epitope) (with BCG); Oncolym (Lym-1 monoclonal antibody); SMART M195 Ab, humanized 13′ 1 LYM-1 (Oncolym). Ovarex (B43.13, anti-idiotypic mouse mAb); 3622W94 mAb that binds to EGP40 (17-1A) pancarcinoma antigen on adenocarcinomas; Zenapax (SMART Anti-Tac (IL-2 receptor); SMART M195 Ab, humanized Ab, humanized); NovoMAb-G2 (pancarcinoma specific Ab); TNT (chimeric mAb to histone antigens); TNT (chimeric mAb to histone antigens); Gliomab-H (Monoclonals—Humanized Abs); GNI-250 Mab; EMD-72000 (chimeric-EGF antagonist); LymphoCide (humanized IL.L.2 antibody); and MDX-260 bispecific, targets GD-2, ANA Ab, SMART IDIO Ab, SMART ABL 364 Ab or ImmuRAIT-CEA. Examples of antibodies include those disclosed in U.S. Pat. No. 5,736,167. U.S. Pat. Nos. 7,060,808, and 5,821,337. Additional embodiments provide the antigen-specific targeting region is CD19-specific targeting region, which includes the sequence of scFv, VH, VL, or a combination thereof of one or more of anti-CD19 antibodies, including but not limited to tafasitamab, loncastuximab tesirine, DI-B4, blinatumomab, coltuximabravtansine, MOR208, MEDI-551, denintuzumabmafodotin, taplitumomabpaptox, XmAb 5871, MDX-1342, and AFM11.

Exemplary CARs, types of genetically engineered cells, and immune checkpoint proteins are described in U.S. Patent Application Publication Nos. 2018-0148508, 2020-0197533, 2019-0358343, and US2021-0095029, all of which are incorporated herein by reference in the entirety. In some embodiments, the genetically engineered cell is a T cell. In other embodiments, the genetically engineered cell is a natural killer (NK) cell, or B cell.

In some embodiments, the extracellular spacer domain comprises a hinge region of an antibody, an Fc fragment of an antibody, a constant domain of heavy chain (CH)2 region of an antibody, a CH3 region of an antibody, or combinations thereof. In some embodiments, the transmembrane domain comprises a transmembrane domain of CD28, a zeta chain of a T cell receptor complex, CD8a, or combinations thereof. In some embodiments, the co-stimulatory domain comprises a signaling domain from any one or more of CD28, CD137 (4-1BB), CD134(OX40), Dap10, CD27, CD2, CD5, intercellular adhesion molecule 1 (ICAM-1), lymphocyte function-associated antigen 1 (LFA-1), Lck, tumor necrosis factor receptor type I (TNFR-I), TNFR-II, Fas, CD30, CD40 and combinations thereof. In some embodiments, the intracellular signaling domain that comprises a signaling domain of one or more of a human CD3 zeta chain, FcγRIII, Fun a cytoplasmic tail of a Fc receptor, an immunoreceptor tyrosine-based activation motif (ITAM) bearing cytoplasmic receptors, and combinations thereof.

VH and VL antibody protein sequences, suitable for the antigen-specific targeting domain of the CAR or for the polypeptide checkpoint inhibitor or the polypeptide binder of TGF-β, are accessible in one or more databases, including the Observed Antibody Space (OAS) database at opig.stats.ox.ac.uk/webapps/oas/.

Additional embodiments provide methods of treating a neoplastic disease in a subject in need thereof, which includes administering to the subject one or more genetically engineered cells disclosed herein, preferably immune cells genetically engineered to express (1) a CAR, (2) a polypeptide that binds an immune checkpoint protein or a polypeptide that binds a ligand of the immune checkpoint protein, and (3) a polypeptide that binds TGF-β or a polypeptide that binds a TGF-β receptor.

In some embodiments, the methods of treating a neoplastic disease further include in vitro culturing of the genetically engineered cells and detecting the presence of a fusion protein in supernatant of the culture, wherein the fusion protein comprises (2) a polypeptide that binds an immune checkpoint protein or a polypeptide that binds a ligand of the immune checkpoint protein, and (3) a polypeptide that binds TGF-β or a polypeptide that binds a TGF-β receptor.

In further embodiments, the secreted fusion protein, e.g., as collected from the culture medium of the genetically engineered cells, is active, i.e., capable of binding a checkpoint protein, blocking a checkpoint protein from binding its cognate binding partner, binding TGF-β or TGF-β receptor, blocking the binding of otherwise free TGF-β and otherwise free TGF-β receptor, or preferably a combination of all these activities. In some embodiments, the activities of the secreted fusion protein are each comparable, e.g., at least 50%, 60%, 70%, 80%, 90%, or 95% compared to a naïve counterpart, e.g., compared to a soluble, recombinant polypeptide checkpoint protein inhibitor and to a soluble naïve TGF-β or TGF-β receptor.

In some embodiments, the methods are for treating a human subject with a cancer. In some embodiments, the subject is one who does not respond to CTLA-4 or PD-1/PD-L1 checkpoint inhibitors. In some embodiments, the subject has melanoma or breast cancer.

In some embodiments, the subject is administered with the genetically engineered cells in combination with another anticancer therapy. Exemplary anticancer therapy include a chemotherapeutic molecule, antibody, small molecule kinase inhibitor, hormonal agent, ionizing radiation, ultraviolet radiation, cryoablation, thermal ablation, or radiofrequency ablation. In other embodiments, the subject is administered in combination with a vaccine.

A “therapeutically effective amount” of the compositions disclosed herein to be administered can be determined by a physician with consideration of individual differences in age, weight, tumor size, extent of infection or metastasis, and condition of the patient (subject). In some embodiments, the therapeutically effective amount of the genetically modified cells is administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The cells can be administered by injection into the site of the lesion (e.g., intra-tumoral injection). In some embodiments, the compositions disclosed herein are administered for one or more exposures, each exposure being 1, 2, 3, 4, 5, 6, 7 days or longer. In some embodiments, the compositions are administered weekly, monthly, or periodically as determined based on progression of the tumor or neoplastic disease, and for a total period of 3 months, 6 months, 1 year, 2 years, 3 years, or longer.

Additional embodiments provide methods of reducing inhibition or exhaustion of T-lymphocytes (T-cells) or improving therapeutic duration or efficacy of cytotoxic T-cells against tumor cells, and the methods include transfecting or transducing the T-cells with a polynucleotide which encodes a fusion protein disclosed herein, and expressing the fusion protein in the T-cells. In some embodiments, the T-cells to be transfected or transduced are ones expressing one or more CARs; and the methods reduces inhibition (e.g., by in vivo tumor environment) or exhaustion of CAR-expressing T-cells. Additional steps of the methods may include isolating, purifying, and/or expanding the T-cells transfected or transduced to express the fusion protein. Subsequently, the T-cells may be infused or administered to a subject in need thereof. Preferably, the T-cells express a CAR with an antigen-specific targeting domain targeting an antigen associated with a neoplastic disease, and the antigen-specific targeting domain has a structure based on the antigen-binding fragment of an antibody. Thus, the methods are suitable for improving therapeutic duration or efficacy or reducing inhibiting or exhaustion of CAR-T cells, which include transfecting the CAR-T cells with a polynucleotide encoding a fusion protein disclosed herein, and expressing the fusion protein in the CAR-T cells, preferably the fusion protein being able to be secreted by the CAR-T cells.

These methods are also suitable for reducing inhibition or exhaustion, or improving therapeutic duration or efficacy, of other immune cells, such as NK cells.

Additional embodiments provide pharmaceutical compositions, which include one or more genetically engineered cells disclosed herewith, and a pharmaceutically acceptable excipient. In some embodiments, a pharmaceutical composition comprises a plurality of the genetically engineered cells, wherein at least 50%, 60%, 70%, 80%, 90% or more of the plurality of the genetically engineered cells express a CAR. In some embodiments, a pharmaceutical composition comprises a plurality of genetically engineered cells that express a CAR but do not express the fusion protein, and a plurality of genetically engineered cells that express a CAR and the fusion protein. In some embodiments, at least 5%, 10%, 20%, 30%, 40%, or 50% or more of the plurality of the genetically engineered cells maintain expression of the CAR after at least one freeze-and-thaw cycle.

Additional pharmaceutical compositions are provided, which include one or more polynucleotides disclosed herein and a pharmaceutically acceptable excipient.

Exemplary pharmaceutically acceptable excipient may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof. Generally a pharmaceutically acceptable excipient is useful in preparing a pharmaceutical composition that is generally safe, non-toxic, and desirable, and includes excipients that are acceptable for veterinary use as well as for human pharmaceutical use.

In various embodiments, the genetically engineered cells according to the invention are in pharmaceutical compositions formulated for delivery via any route of administration. “Route of administration” may refer to any administration pathway known in the art, including but not limited to parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. In some embodiments, the genetically engineered cells according to the invention are in pharmaceutical compositions formulated for intra-tumoral injection. Via the parenteral route, the compositions may be in the form of solutions or suspensions for infusion or for injection.

In various embodiments, the polynucleotides and/or vectors of the invention are in pharmaceutical compositions formulated for delivery via any route of administration. For example, the pharmaceutical compositions comprising the polynucleotides and/or vectors and pharmaceutically acceptable excipients are administered via an aerosol, nasal, oral, transmucosal, transdermal or parenteral route. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection. Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

Further embodiments provide vectors for transduction or transfection of cells with a polynucleotide disclosed herein. In some embodiments, the vector is a viral vector, e.g., a lentiviral vector or a retroviral vector. In other embodiments, the vector is a non-viral vector.

Methods for making the afore-mentioned genetically engineered cells, or polynucleotides and/or vectors, are also described herein. The fusion constructs, with or without being operably linked to a nucleic acid sequence encoding a chimeric antigen receptor, are generated at the DNA level; and the resulting DNAs are integrated into expression vectors, and expressed in cells to produce the genetically engineered cells of the invention. Optionally the method further includes a step of isolating, purifying, and/or proliferating the genetically engineered cells of the invention, and/or detecting the fusion protein from the host cell culture.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

Generation and Characterization of CAR-T Cells and Target Cell Lines

The schematic representation of the retroviral vector constructs used in this study is shown in FIG. 1B. Based on the construct of the second-generation anti-human CD19 CAR (CD19 CAR) that contains an anti-human CD19 scFv, a hinge and transmembrane domain, an intracellular CD28 costimulatory domain and a CD3 zeta activation domain, we generated the anti-CD19 CAR with anti-PD1 scFv secretion (CD19 CAR-αPD1) by using a P2A element as the linker between the CD19 CAR sequence and the anti-PD1 scFv sequence. The feasibility and functionalities of CD19 CAR-αPD1 have been demonstrated by various in vitro and in vivo experiments in our previous study. In this study, we further engineered the anti-CD19 CAR with trap protein secretion (CD19 CAR-Trap) by fusing the anti-PD1 scFv sequence with a TGF-β binding sequence derived from TGF-βRII through a glycine-serine (GS) linker.

Human peripheral blood mononuclear cells (PBMCs) were activated and transduced with each of the three CAR constructs. As shown in FIG. 1C, CARs were expressed in primary lymphocytes at a similarly high level (>60%). During the two-week T cell expansion phase, the CAR expression levels were stably maintained. After performing freeze and thaw, about 40% of the CAR expression was maintained. To test the antigen-specific functionalities of CAR-T cells, we engineered three different target cell lines, SKOV3-CD19, H292-CD19, and PC3-PD-L1-CD19 (also referred as PC3-CD19). The three target cell lines showed similar expression levels of antigen CD19 and TGF-β (FIG. 1E, 1F), but for immune checkpoint molecule PD-L1, PC3-CD19 had a significantly higher expression level than the other two cell lines (FIG. 1D). Since the cell line with a high expression of TGF-β and PD-L1 might serve as a better tool to test the effect of the bifunctional trap protein on CAR-T cells, PC3-CD19 was used in most of the following experiments.

Characterization of Trap Protein

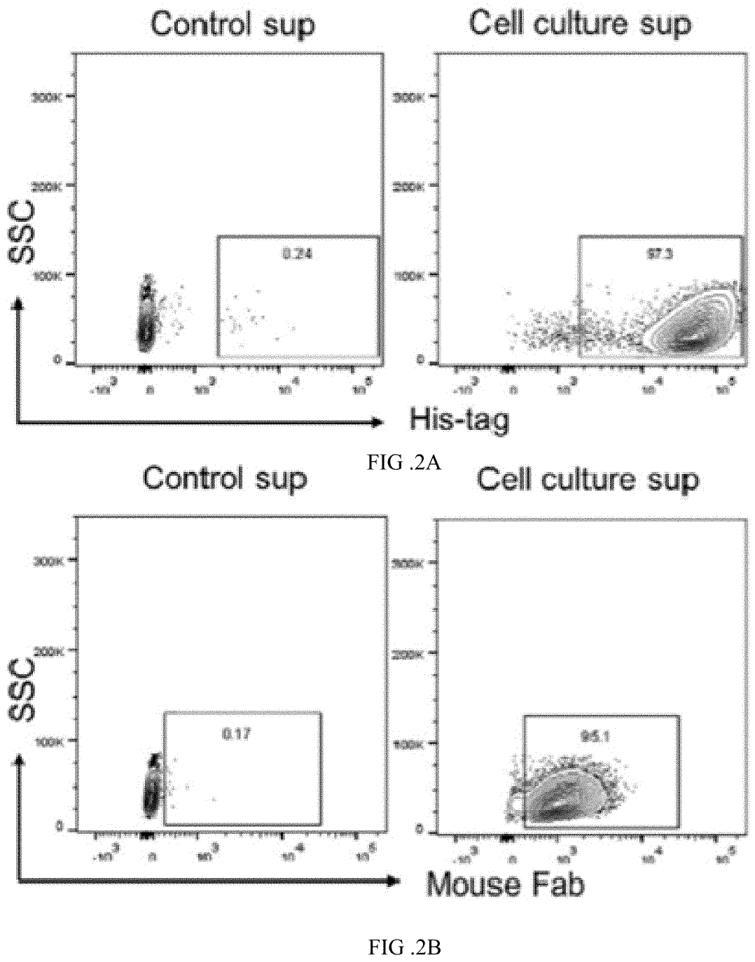

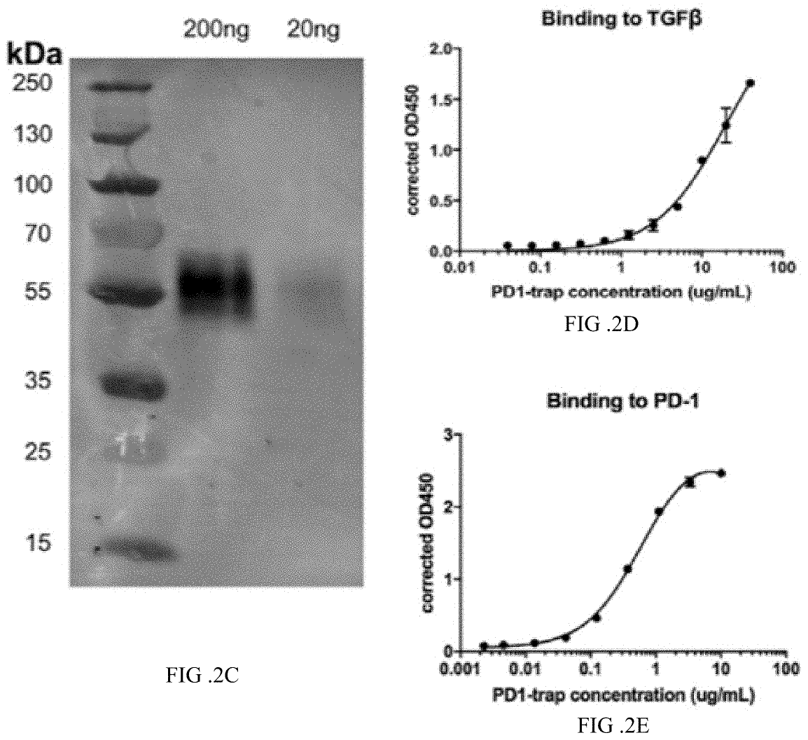

To assess trap protein synthesized by engineered cells, 293T cells were transfected to produce trap protein labeled with a His-tag. Three days post-transfection, the cell culture supernatant was harvested and used to incubate 293T cells engineered to express PD-1 on the cell surface. After 1 hour of incubation, trap protein binding to 293T-PD-1 was detected by either anti-His-tag antibody or anti-mouse F(ab′)2 antibody (FIG. 2A, 2B).

Furthermore, trap protein was purified from cell culture supernatant of transfected 293T cells using a His-tag-labeled protein purification protocol. Western blotting analysis confirmed the secretion of trap protein with molecular weight of 56 kDa (FIG. 2C). The bifunctional binding activity of trap protein to PD-1 and TGF-β was confirmed by enzyme-linked immunosorbent assay (ELISA), wherein purified protein was added to recombinant human PD-1-Fc- or TGF-β-coated plates at different concentrations, and the binding was detected by anti-His-tag antibody (FIG. 2D, 2E).

Trap Protein Secretion Changes Cytokine Expression and Improves Proliferation of CAR-T Cells in Response to Antigen Stimulation

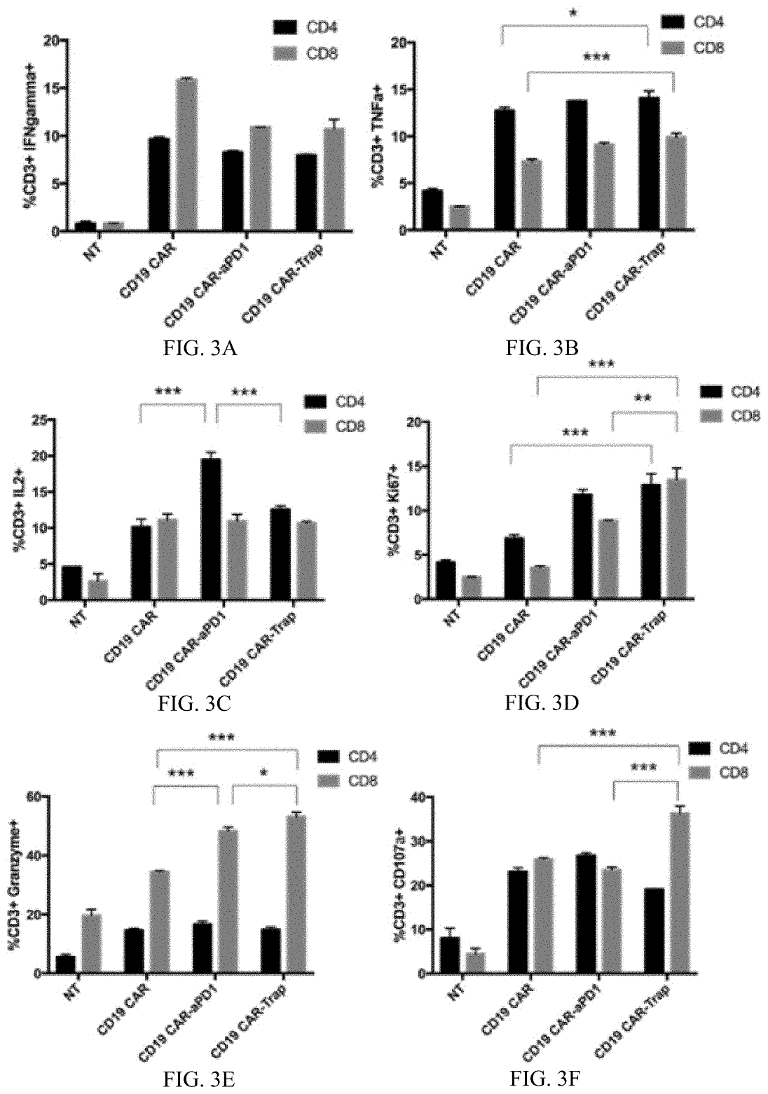

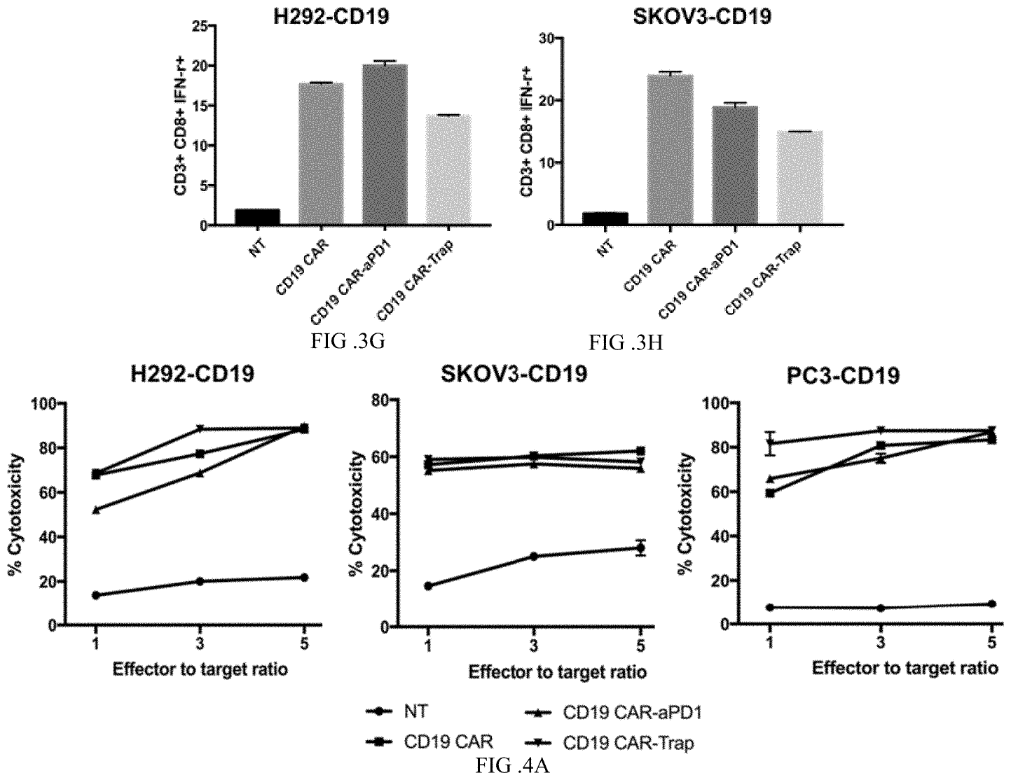

To study how trap protein affects the response of CAR-T cells to target cells in vitro, we co-cultured CAR-T cells with PC3-CD19 cells at a 1:1 cell-to-cell ratio for 16 hours in the presence of protein transport inhibitor. Compared with the non-transduced (NT) control cells, CAR-′I′ cells all responded to the stimulation from target cells with increased expression of the pro-inflammatory cytokines interferon (IFN)-γ, tumor necrosis factor (TNF)-α, and interleukin (IL)-2. However, the effects of protein drug secretion on the cytokines were quite different. For. IFN-γ, CD19 CAR-αPD-1 and CD19 CAR-Trap didn't exhibit a higher percentage of IFN-γ⁺ T cells than CD19 CAR in either CD4⁺ or CD8⁺ T cell populations (FIG. 3A). For TNF-α, CD19 CAR-Trap showed more production than CD19 CAR in both CD4⁺ or CD8⁺ T cell populations, whereas CD19 CAR-αPD-1 only had an effect similar to that of the CD4⁺ T cell population (FIG. 3B). When IL-2 was examined, no difference was detected with the exception of the CD19 CAR-αPD-1 group, which exhibited a higher expression in CD4⁺ T cell population compared with the other two groups (FIG. 3C). Besides PC3-CD19, the immune response of CAR-′T cells upon stimulation from H292-CD19 and SKOV3-CD19 was confirmed by intracellular IFN-γ staining (FIG. 3G, 3H).

Granzyme B and CD107a expression were measured to assess the cytotoxic function of CAR-T cells upon antigen stimulation. Unlike the results of pro-inflammatory cytokine production, CD19 CAR-Trap showed a consistently enhanced expression of granzyme B and CD107a in its CDS8⁺ T cells population in all four groups (FIG. 3E, 3F), CD19 CAR-αPD-1 had an improved production of granzyme B than CD19 CAR in CD8 T cells population, but no significant improvement was observed in the production of CD107a (FIG. 3D, 3E).

Meanwhile, the expression of Ki67 in CAR-T cells was measured to elucidate the cell proliferative potential. Among three CAR-T groups, CD19 CAR-Trap had the highest positive rate of Ki67 in both CD4⁺ or CD8⁺ T cell populations, followed by CD19 CAR-αPD-1 (FIG. 3D).

Taken together, the results clearly show that trap protein secretion differentially influenced T cell functional markers. Although trap protein didn't profoundly affect pro-inflammatory cytokines expression, it significantly enhanced the expression of cell proliferation marker and cytotoxicity-related molecules, which indicates that upon short-term stimulation, CAR-T cells with trap protein secretion can proliferate faster and exert a more potent target killing effect.

Trap Protein Secretion Attenuates TGF-β Signaling, Reduces the Proportion of Treg, and Improves the Effector Cytokine Secretion of CAR-T Cells

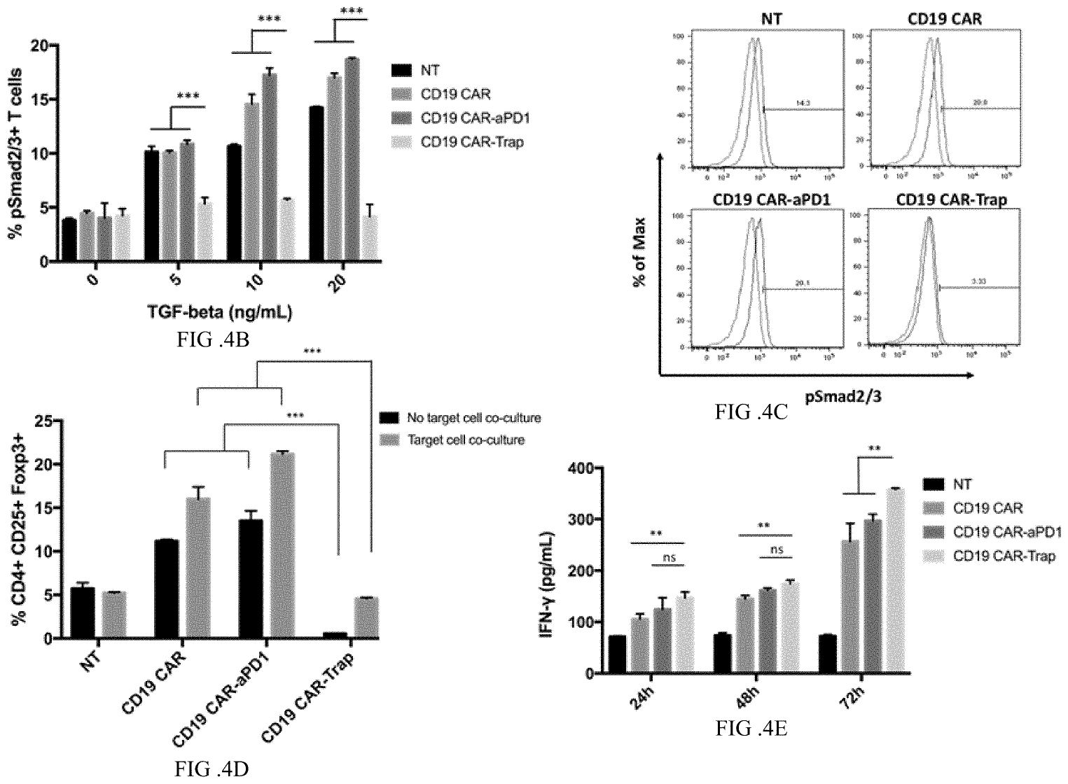

We next sought to study the capability of CAR-T cells with trap protein secretion to lyse target cells by co-culturing, CAR-′I′ cells with a series of target cells at various effector-to-target ratios for 24 hours. In the co-culture with H292-CD19, SKOV3-CD19 and PC3-CD19 cells, at different effector-to-target ratios, CD19 CAR-Trap all exhibited a cell killing capability comparable to that of CD19 CAR and CD19 CAR-αPD-1 (FIG. 4A). The antigen specific cell lysis of the CAR-T cells was potent that the saturation was observed at the effector-to-target ratio of 3:1 for H292-CD19 and PC3-CD19 cells, and at 1:1 for SKOV3-CD19 cells (FIG. 4A). The effect of trap protein secretion on cell killing capability was, however, not significant within 24 hours of co-culture.

In human CD4⁺ T cells, antigen stimulation, concomitant with TGF-β induces the expression of Foxp3 in naïve CD4⁺ T cells and converts them to CD4⁺CD25⁺Foxp3⁺ Tregs. To confirm the ability of trap protein to block the TGF-β signaling pathway, TGF-β-induced phosphorylation of Smad2/3 and Foxp3 expression in human T cells was measured. When CAR-T cells were incubated with TGF-β at various concentrations for 24 hours, the phosphorylation of Smad2/3 was elevated in the NT, CD19 CAR, and CD19 CAR-αPD-1 groups in a dose-dependent manner, whereas that in the CD19 CAR-Trap group remained at the basal level (FIG. 4B, 4C). The expression of Foxp3 in the CD4⁺ T cell population, representing the differentiation of Tregs, was measured with or without co-culture with PC3-CD19 cells. We found that CD19 CAR and CD19 CAR-αPD-1 exhibited a greater proportion of Tregs compared with NT, especially after being co-cultured with target cells. However, CD19 CAR-Trap contained fewer Tregs than CD19 CAR and CD19 CAR-αPD-1, irrespective of co-culture with target cells (FIG. 4D). Notably, the proportion of Tregs in CD19 CAR-Trap T cells was lower than that in NT cells.

To evaluate the effect of trap protein on effector function, CAR-T cells were co-cultured with PC3-CD19 for different durations. The conditioned supernatants were collected and processed for IFN-γ quantification by ELISA. Upon stimulation by PC3-CD19 for 24 hours and 48 hours, CD19 CAR-αPD1 and CD19 CAR-Trap secreted a similar amount of IFN-γ, which was higher than the amount secreted by CD19 CAR (FIG. 4E). After 72 hours of antigen stimulation, CD19 CAR-Trap had a significantly higher secretion of IFN-γ than either CD19 CAR or CD19 CAR-αPD-1 (FIG. 4E).

Trap Protein Secretion Rescues the CAR-T Cell from Exhaustion by Limiting the Up-Regulation of Immune Checkpoint Molecules

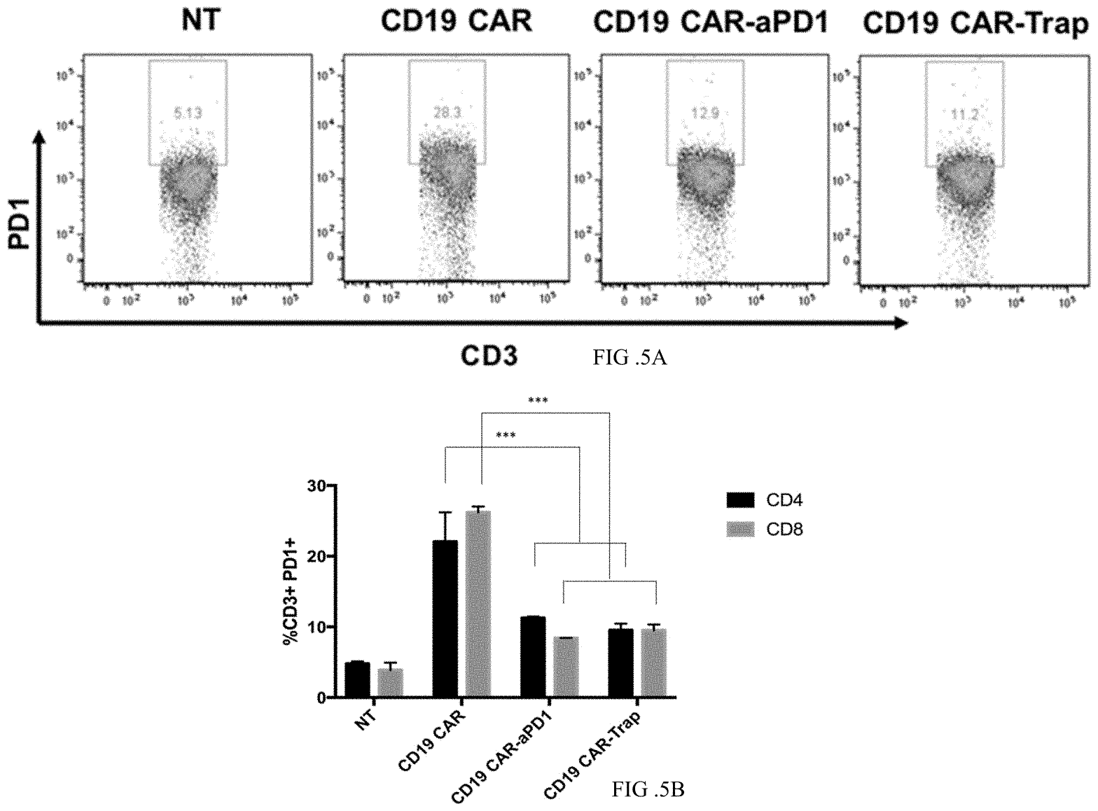

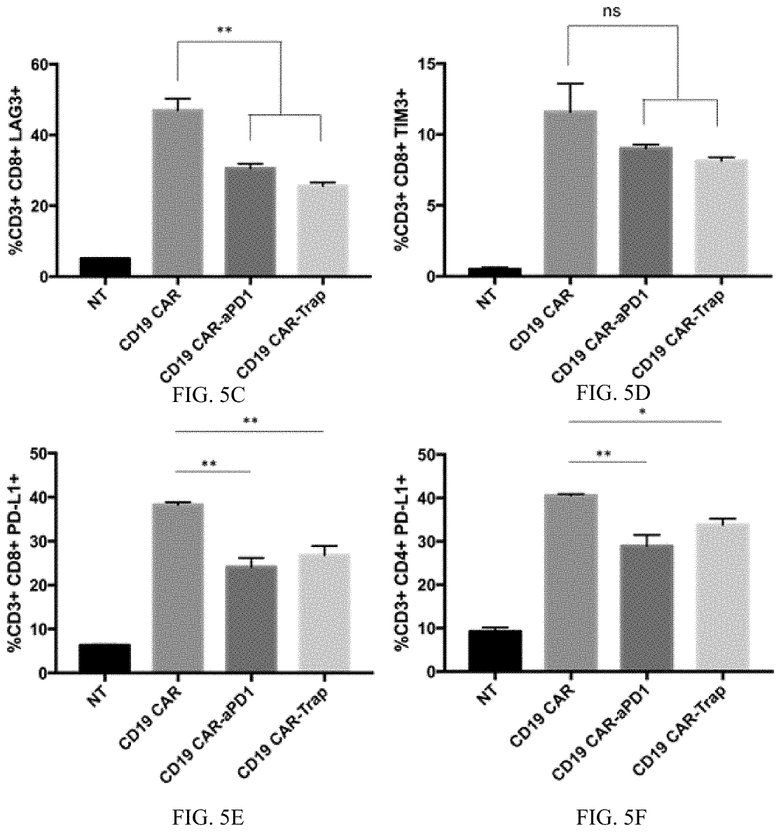

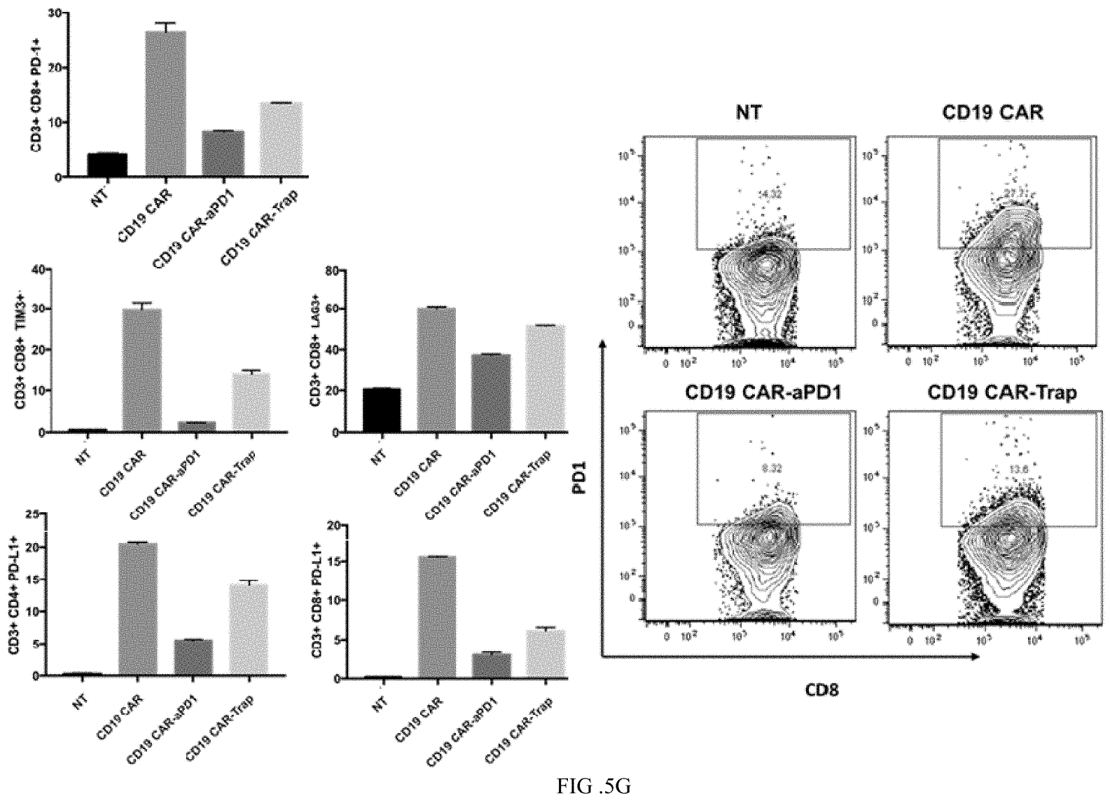

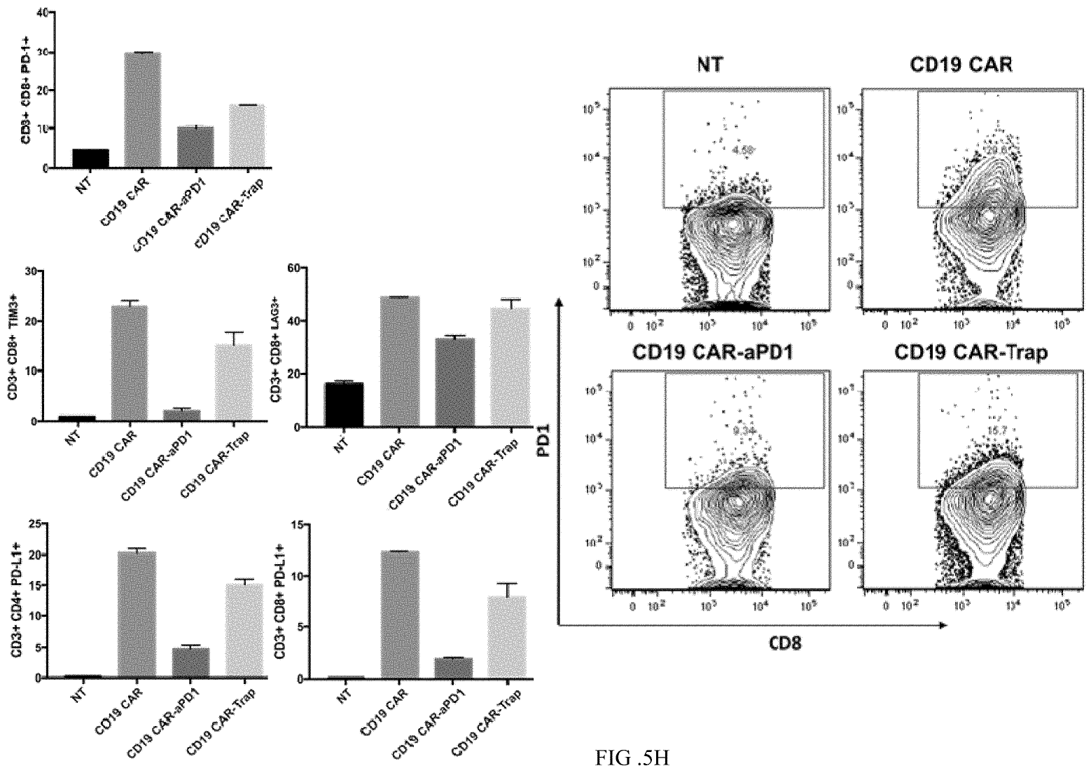

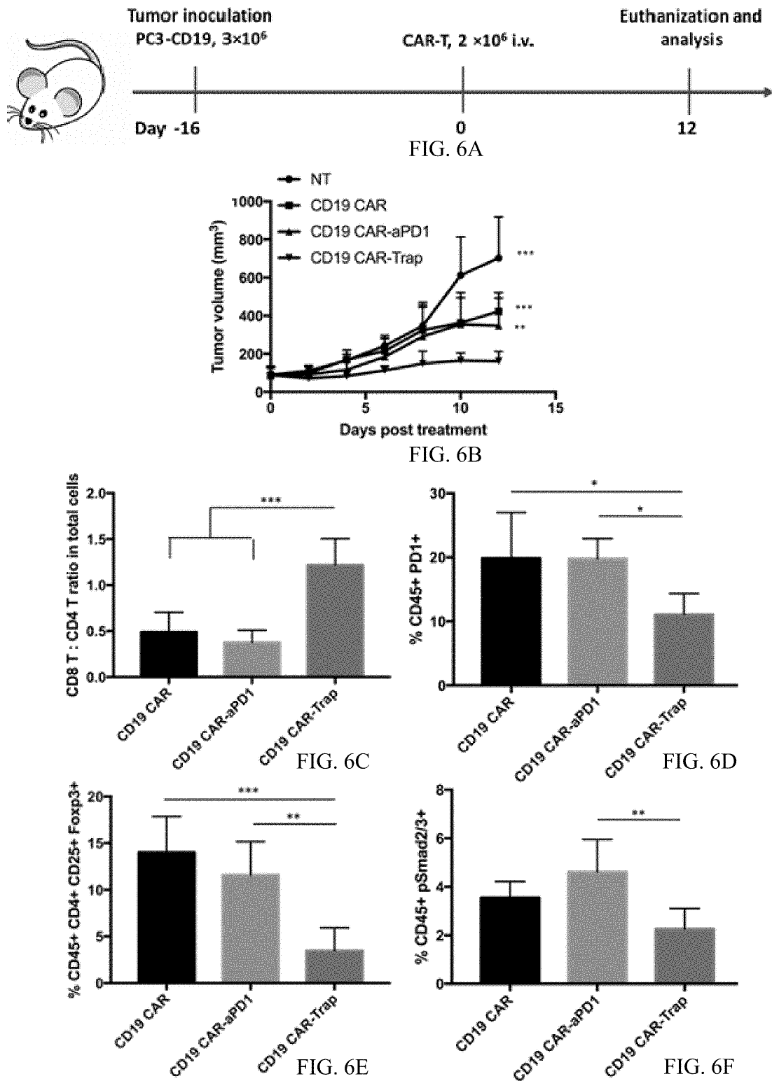

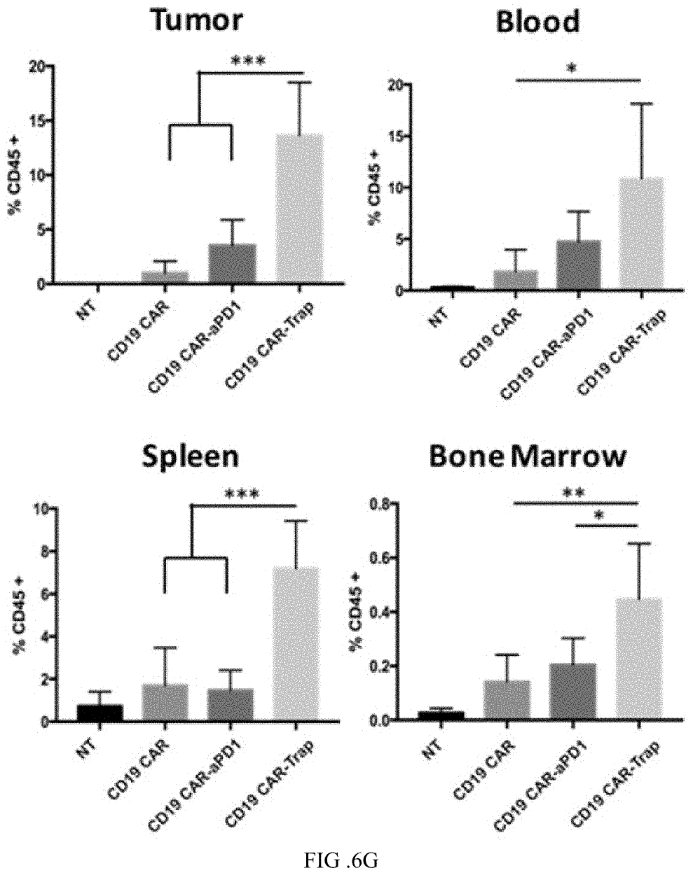

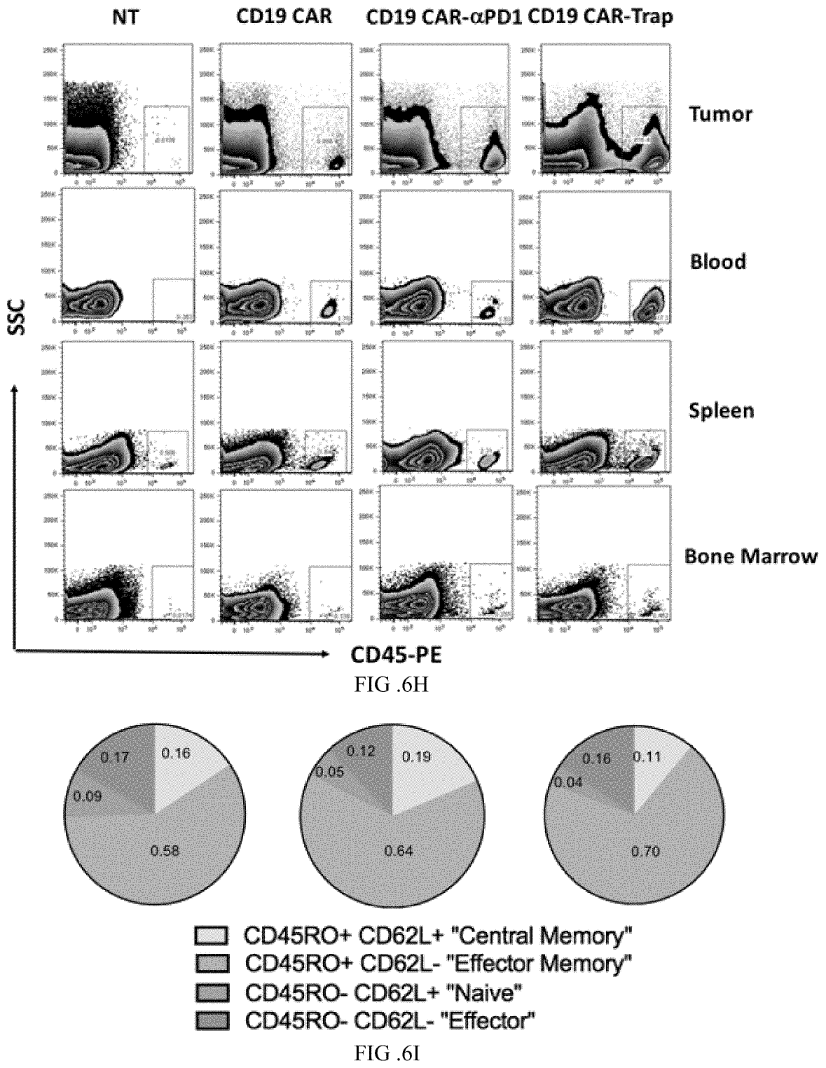

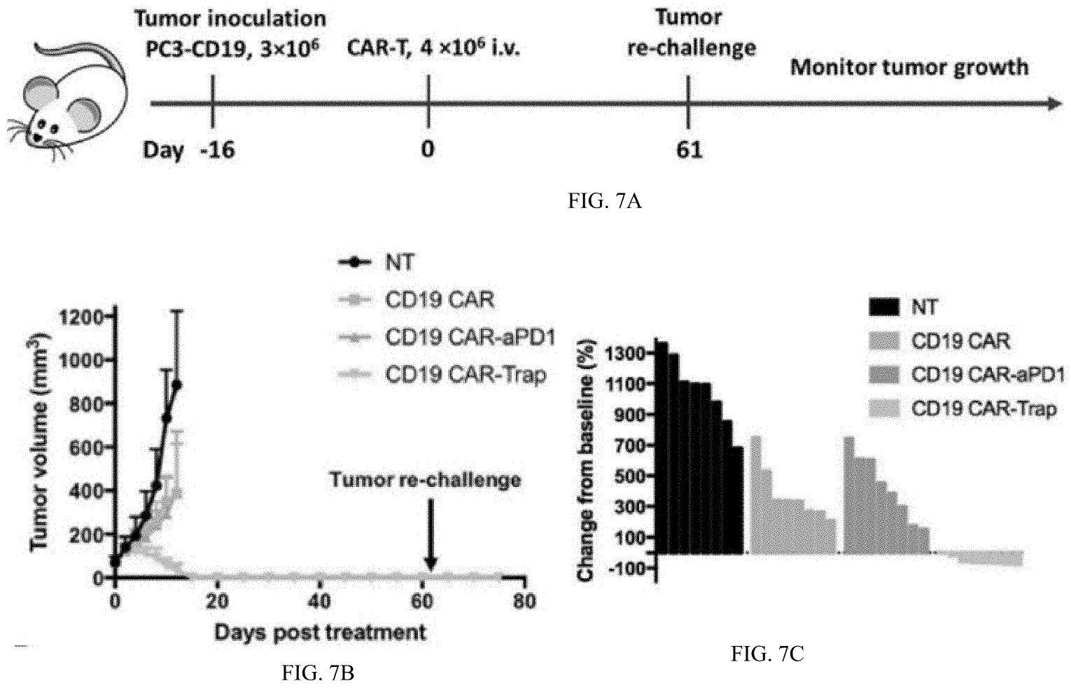

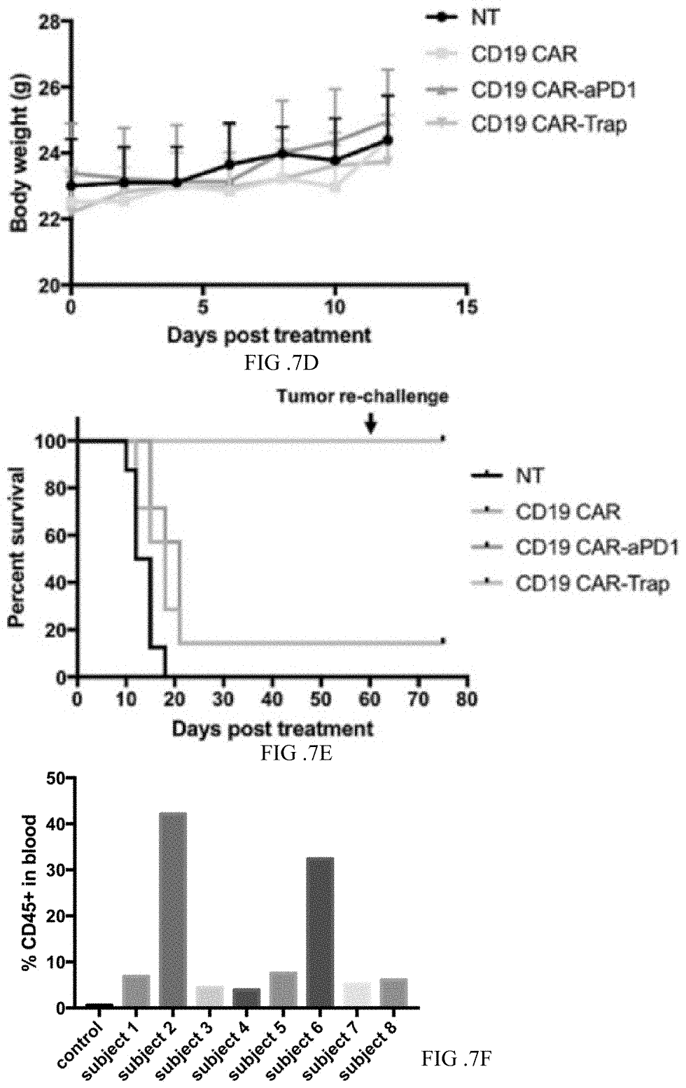

Importantly, the trap protein blocks the binding between PD-1 and PD-L1, concomitant with the blocking of TGF-β signaling, as a step to remediate cell exhaustion. Since PD-1/PD-L1 binding induces increased expression of PD-1 on the T cell surface, we investigated the effect of trap protein on PD-1 expression by co-culturing CAR-T cells with PC3-CD19 cells. After 24 hours, all CAR T groups exhibited upregulated PD-1 expression compared with NT. In contrast, PD-1 expression was significantly lower in the CD19 CAR-αPD-1 and CD19 CAR-Trap groups compared with that in the CD19 CAR group, indicating that the secreted protein drugs, anti-PD1 scFv and trap protein, played a role in limiting the upregulation of PD-1 expression. No significant difference was observed between CD19 CAR-αPD-1 and CD19 CAR-Trap (FIG. 5A). When different T cell subtypes were examined, similar results were observed in both CD4⁺ T cells and CD8⁺ T cells (FIG. 5B).

In addition to PD-1, there are other immune checkpoint molecules expressed on T cell surface, such as lymphocyte-activation gene 3 (LAG3), T cell immunoglobulin domain and mucin domain-containing protein 3 (TIM3), and PD-L1, contributing to T cell exhaustion upon antigen stimulation as a complex interaction network. To further evaluate the capability of trap protein to rescue CAR-T cells under these conditions, we tested the expression of LAG3, TIM3, and PD-L1 in CAR-T cells after the co-culture with PC3-CD19 cells for 24 hours. Again, CAR-αPD-1 and CD19 CAR-Trap had less LAG3 and PD-L1 upregulation than that exhibited by CD19 CAR (FIG. 5C, 5E, 5F). In the analysis for TIM3, CAR-αPD-1 and CD19 CAR-Trap showed a trend toward the inhibition of TIM3 upregulation, but it didn't reach statistical significance (FIG. 5D). The effect of trap protein to alleviate CAR-T cell exhaustion was also confirmed by the data collected from co-culture with H292-CD19 and SKOV3-CD19. (FIG. 5G, 5H).

Trap Protein-Secreting CAR-T Cells Exhibit Enhanced Expansion, Infiltration, and Antitumor Efficacy In Vivo

Having confirmed the in vitro specificity and functionality of CD19 CAR-Trap in response to CD19⁺PD-1⁺ target cells, we continued to evaluate the antitumor potential of CD19 CAR-Trap cells in vivo utilizing a subcutaneous human prostate cancer xenograft model in NOD.Cg-Prkdc^{scid}IL2^{tm1Wj1}/Sz (NSG) mice. The experimental procedure for animal study is shown in FIG. 6A.

Briefly, PC3-CD19 cells (3×10⁶) were injected into the right flank of NSG mice. When the average tumor size reached 75-100 mm³ on day 16 post tumor inoculation, the tumor bearing mice were randomized into four groups (5 mice per group), and received 2×10⁶ CAR-T cells through intravenous injection. During the 12-day tumor growth monitoring period, groups receiving CAR-T treatment showed more efficacy in inhibiting tumor progression compared to the NT group. Among CAR-T treatment groups, CD19 CAR-Trap exhibited significantly slower tumor growth than CD19 CAR and CD19 CAR-αPD-1, indicating enhanced antitumor efficacy (FIG. 6B). In comparison between CD19 CAR and CD19 CAR-αPD-1, no significant difference was observed.

On day 12 post-treatment, mice were all euthanized, and T cells in tumors were analyzed. In the analysis of tumor samples, T cell infiltration of the NT group was negligible; therefore we only focused on the three CAR-T treatment groups. In terms of tumor-infiltrating lymphocytes (TILs), the CD19 CAR-Trap group had a higher CD8 T:CD4 T ratio than either the CD19 CAR group or the CD19 CAR-αPD-1 group (FIG. 6C). Consistent with the data from in vitro studies, treatment with CD19 CAR-Trap resulted in inhibition of PD-1 expression on TILs, differentiation of Foxp3⁺ Tregs, and TGF-β signaling (FIG. 6D, 6E, 6F). Furthermore, TILs of the CD19 CAR-Trap treatment group displayed a slightly higher proportion of effector memory T cells than either the CD19 CAR group or the CD19 CAR-αPD-1 group (FIG. 6I).

To compare the CAR-T infiltration and expansion in vivo, tumor, blood, spleen, and bone marrow of each group were harvested and examined. The T cell population was identified using tissue samples from a non-treated mouse as a control (FIG. 6H). We found that CD19 CAR-Trap maintained a high proportion of T cells (14%) in tumors on day 12 post-treatment, which was more than three times of the T cell percentages shown in CD19 CAR group (1%) and CD19 CAR-αPD-1 group (4%) (FIG. 6G). Similar results were shown in the analysis for blood, spleen, and bone marrow. Compared with NT, all CAR-T treatment groups had more T cells present, but CD19 CAR-Trap exhibited a significantly higher level than other CAR-T groups (FIG. 6G). In a nutshell, within 12 days, CD19 CAR-Trap showed a superior expansion in tumor, blood, spleen. and bone marrow.

Trap Protein-Secreting CAR-T Cells Achieve Long-Term Remission and Prevent Tumor Relapse

To further explore the antitumor potential of CD19 CAR-Trap, we conducted a long-term in vivo efficacy study with the dose of CAR-T cells doubled (4×10⁶ cells). The establishment of the tumor model was the same as previously described. As shown in FIG. 7A, tumor-bearing mice were randomized into four groups (8 mice per group) with an average tumor size of ˜100 mm³. Most mice of the NT group reached the endpoint tumor size of 1000 mm³ within the 12 days. Although CD19 CAR and CD19 CAR-αPD-1 indistinguishably slowed the tumor growth rate, CD19 CAR-Trap decreased the tumor size by more than 50% (FIG. 7B). Notably, we found that tumors of the mice in CD19 CAR-Trap group shrank faster when the tumor volume got smaller, and they could not be detected at 16 days post treatment. In the meantime, no significant body weight loss was observed in any treatment group (FIG. 7C).

After taking out the mice that met endpoint, we continued to monitor the survival of mice for 60 days. The 8 mice of the CD19 CAR-Trap group kept complete remission until the end. In the CD19 CAR and CD19 CAR-αPD-1 groups, only 1 mouse achieved complete remission. Compared with either CD19 CAR (12.5%) or CD19 CAR-αPD-1 (12.5%), CD19 CAR-Trap significantly improved the survival rate (100%) (FIG. 7D, 7E). The 8 mice of CD19 CAR-Trap group were then bled on day 61 for T cell analysis. Except for 2 mice found to develop graft-versus host disease (GvHD) with an abnormally high percentage of T cells in blood and GvHD symptoms, 6 mice had percentages of T cells that ranged from 3% to 8% (FIG. 7F). We injected those 6 mice with 1×10⁶ PC3-CD19 cells into the left flank subcutaneously and monitored them for another two weeks. None of the 6 mice developed detectable tumors.

Over the years, despite the unprecedented success of CAR-T therapy has achieved in hematological malignancies, the clinical results of it in solid tumor treatment are still far from satisfactory. Unlike hematological malignancies, the unique tumor microenvironment (TME) poses significant challenges for CAR-T cells to mount their effector function and expand at the tumor site. PD-1/PD-L1 pathway and TGF-β signaling pathway have been demonstrated as the two major resistance mechanisms exerted by solid tumors in response to T cell antitumor activity. In clinical studies, single agents targeting only one signaling pathway are unable to induce effective immune responses in most patients. To assess the potential of CAR-T cells with trap protein secretion to solve these issues, an ideal tumor model should have three characteristics: (1) overexpression of tumor-associated antigen; (2) a high expression level of PD-L1 (3) a high secretion level of TGF-β. Thus, we chose the prostate cancer cell line PC3, known to have high endogenous TGF-β secretion, and we engineered it to overexpress PD-L1 and CD19, documented antigen in CAR-T research. The engineered PC3-PDL1-CD19 (termed PC3-CD19), together with engineered ovarian cancer cell line SKOV3-CD19 and lung cancer cell line H292-CD19, as target cell lines, provided the basis for our in vitro and in vivo analyses.

Numerous studies have shown that activation of the PD-1/PD-L1 pathway in T cells inhibits cytokine production, T cell proliferation and effector function. In our previous study, the secretion of anti-PD-1 scFv was demonstrated to help CAR-T cells maintain effector function and rescue CAR-T cells from exhaustion in vitro. In this study, to investigate the effect of a secreted trap protein, we conducted similar co-cultures between CAR-T cells and the target cell lines, and we found that CAR-T cells with trap protein secretion showed significantly improved cytotoxic function by elevated granzyme B and CD107a expression and higher proliferation potential by higher Ki67 expression in comparison with the parental CAR-T cells. For effector cytokine IFN-γ, a higher secretion level from trap protein self-secreting CAR-T cells was observed, especially when co-cultured with target cells for a long period. In these analyses, anti-PD1 self-secreting CAR-T cells showed a similar, but weaker, enhancement. The different results between anti-PD-1 self-secreting CAR-T and trap protein self-secreting CAR-T might result from the blocking effect of trap protein against the TGF-β signaling pathway. Dimeloe et al. reported that tumor-derived TGF-β inhibits IFN-γ production by human CD4⁺ T cells, but that the production capacity can be restored by applying TGF-β-neutralizing antibodies. In addition to inhibition of T cell proliferation, TGF-β also inhibits the expression of perforin, granzyme A, granzyme B, and IFN-γby CD8⁺ T cells. In the staining for CAR expression, we noticed that the self-secreting CAR-T cells had a lower CAR density than the parental CAR-T cells, probably because the insertion of additional DNA sequences, which encode anti-PD-1 scFv or trap protein, impairs vector production and transduction efficiency. However, the cytotoxic effector functions of self-secreting CAR-T cells were not comprised, as indicated by our in vitro and in vivo results.

Upon persistent antigen stimulation, the expression level of PD-1 in T cells is upregulated. To study the ability of trap protein to overcome suppression posed by immune checkpoints, we analyzed the expression of PD-1 and its co-expressing immune checkpoint molecules, such as LAG3, TIM3, and PD-L1, in CAR-T cells post-antigen stimulation. The self-secreting CAR-T cells alleviated upregulation of PD-1, LAG3, TIM3 and PD-L1 after co-culture with various target cell lines. Positive correlations among the expressions of PD-1, TIM3, and LAG3 have been reported. Our data indicate that the secreted trap protein alters the signaling network of immune checkpoint molecules, rather than merely targeting the PD-1/PD-L1 pathway. The inhibitory effect of trap protein on the expression of LAG3, TIM3 and PD-L1 might then contribute to the enhanced functions of trap protein self-secreting CAR-T cells, as well.

On the other hand, BOTH autocrine and paracrine TGF-β in the TME promote differentiation of Tregs and attenuate the activation of T cells through the induction of Smad2/3 phosphorylation and Foxp3 expression. In the incubation with recombinant TGF-β, we found the phosphorylation of Smad2/3 in conventional CAR-T cells and anti-PD-1 self-secreting CAR-T cells increased with the TGF-β concentration, whereas trap protein self-secreting CAR-T cells remained at the basal level, indicating that trap protein allows for the CAR-T cells to counteract TGF-β-mediated signaling. Apart from tumor cell-derived TGF-β, it has been reported that T cell-derived TGF-β also regulates T cell differentiation and tolerance. However, it is not known if the T cell-derived TGF-β will be increased in genetically modified CAR-T cells. We noticed that even without antigen stimulation, conventional CAR-T cells and anti-PD-1 self-secreting CAR-T cells had an elevated percentage of Tregs compared with non-transduced T cells. Protected by the inhibition of TGF-β signaling pathway, trap protein self-secreting CAR-T cells kept a small Treg proportion with or without antigen stimulation.

The superior antitumor efficacy of trap protein self-secreting CAR-T cells was demonstrated in the PC3-CD19 xenograft mouse model. At a low dose of 2 million CAR-T cells, trap protein self-secreting CAR-T cells achieved stability of tumor growth in the treated mice, whereas other treatments failed to control the tumor progression. The undifferentiated efficacy of conventional CAR-T and anti-PD-1 self-secreting CAR-T cells indicates that targeting only the PD-1/PD-L1 pathway is not enough to enhance CAR-T cells in this progressive prostate tumor model. CD8⁺/CD4⁺ T cell ratio at the tumor site is another important prognostic marker in immunotherapy. Research has found that, in solid tumors, a high CD8⁺/CD4⁺ T cell ratio is often correlated with absence of metastasis, slow tumor progression and improved survival. In our in vivo results, the CD8⁺/CD4⁺ T cell ratio of trap protein self-secreting CAR-T group was twice that of the conventional CAR-T group and anti-PD-1 self-secreting CAR-T group, indicating trap protein self-secreting CAR-T cells are skewed toward an effector phenotype, thereby possibly contributing to the enhanced inhibition of tumor progression. Although CD4⁺ T cells are not the major T cell population within the tumor-infiltrating T cells, they contribute to overall antitumor efficacy via multiple mechanisms in the presence of trap protein that can block TGF-β signaling. Recent studies have shown that TGF-β blockade not only increases IL-2 and IFN-γ secretion of CD4⁺ T helper type I (Th1) cells but also improves IL-4 expression of CD4⁺ Th2 cell, causing reorganization of tumor vasculature, tumor tissue hypoxia, and the consequent tumor cell death. For the expressions of biomarkers related to PD-1/PD-L1 and TGF-β pathways, we found consistency between in vivo and in vitro data. Trap protein self-secreting CAR-T group exhibited lower expression of PD-1, attenuated phosphorylation of Smad2/3, and a smaller proportion of Foxp3+ Tregs. Taken together, these findings can help to explain why the trap protein self-secreting CAR-T group exhibits dramatically improved antitumor efficacy and T cell expansion in tumor, spleen, and bone marrow.

In the subsequent in vivo study, with the treatment protocol slightly modified by doubling the dose of CAR-T cells, we focused on the capability of trap protein self-secreting CAR-T cells to eradicate solid tumors and to improve survival. Within 12 days, the change in dose showed no significant impact on the tumor growth of conventional CAR-T group and anti-PD-1 self-secreting CAR-T group; it did lead to a remarkable tumor shrinkage in the trap protein self-secreting CAR-T group. Trap protein self-secreting CAR-T cells successfully eradicated tumors on day 16 post-treatment and improved survival rate compared with the conventional CAR-T group and anti-PD-1 self-secreting CAR-T group. It is worth noting that, for 6 out of the 8 mice in trap protein self-secreting CAR-T group, CAR-T cells can persist in body for 60 days without causing graft-versus-host disease. When tumor rechallenge was given by injecting tumor cells into the cured mice, no new tumor was developed, which indicates that the remaining trap protein self-secreting CAR-T cells in the mouse body prevent tumor recurrence.

Further modifications can be made with the trap protein self-secretion strategy demonstrated herein to expand CAR-T therapy in different clinical settings. First, the antigen CD19 used in this proof-of-concept study is not a solid tumor antigen. Solid tumor antigens, such as prostate-specific membrane antigen (PSMA) or human epidermal growth factor receptor 2 (HER2), are conceived as targets for the antigen-specific domain of the CAR. Second, given the numerous dynamic interplays between cells and molecules in the TIME, the simultaneous inhibition of PD-1/PD-L1 and TGF-β signaling pathways may even have a more profound effect than the results of our in vivo study might suggest. Therefore, it would be beneficial to unveil the antitumor potential of the trap protein self-secreting CAR-T cells in syngeneic mouse models. Finally, this self-secretion platform is modular and flexible, and it can be applied to combinations of different antibodies and binding sequences for receptors or ligands. For example; the combinations of anti-PD-L1 antibody or anti-cytotoxic T-lymphocyte-associated protein 4 (CTLA-4) antibody with TGF-β can also be explored.

Cell Lines

SKOV3 ovarian cancer cell line, PC3 prostate cancer cell line and 293T were obtained from ATCC. NCI-H292 lung cancer cell line was kindly provided by Dr. Ite Laird-Offringa. The PC3 cell line was transduced with viral vector to express a high level of PD-L1 on cell surface as PC3-PD-L1 and sorted to 99% purity. The SKOV3, NCI-H292 and PC3-PD-L1 cell lines were transduced with a lentiviral vector FUW-CD19. The transduced cells were then stained with anti-human CD19 antibody and sorted to 99% purity. The cell culture methods for 293T, engineered SKOV3-CD19 and H292-CD19 were described in our previous publications. PC3 and PC3-PD-L1-CD19 (also referred as PC3-CD19) were maintained in D10 medium consisting of DMEM supplemented with 10% fetal bovine serum (FBS), 2 mM L-glutamine, 100 U/mL penicillin and 100 μg/mL streptomycin.

Plasmid Construction

The retroviral vectors encoding anti-CD19 CAR (CD19 CAR) and anti-CD19 CAR with anti-PD-1 scFv secretion (CD19 CAR-αPD-1) were constructed based on the MP71 retroviral vector kindly provided by Prof. Wolfgang Uckert, as described in Clin. Cancer Res. 23, 6982-6992, 2017. The vector encoding anti-CD19 CAR with trap protein secretion (CD19 CAR-Trap) was generated based on CD19 CAR-αPD-1. The RV-CD19 CAR-Trap vector consisted of the following components in frame from 5′ end to 3′ end: the MP71 retroviral backbone, a NotI site, the anti-CD19 CAR, a T2A sequence, a human IL-2 leading sequence, the anti-PD1 scFv light chain variable region, (GGGGS)₃ (SEQ ID NO:20), the anti-PD1 scFv heavy chain variable region, (GGGGS)₂ (SEQ ID NO:19), a TGF-β-binding sequence, and an EcoRI site. Another trap sequence with the same components followed by a His-tag sequence was inserted into pcDNA3.1 vector for trap protein purification and binding assessment.

The anti-PD1 scFv in the CD19 CAR-Trap vector was derived from the amino acid sequence of monoclonal antibody 5C4-specific against human PD-1. The TGF-β-binding sequence was derived from the amino acid sequence of the ligand binding region in human TGF-βRII extracellular domain. The amino acid sequences were codon optimized with online codon optimization tool and corresponding DNA sequences were synthesized by Integrated DNA Technologies. The DNA sequence encoding trap protein was ligated into the CD19 CAR vector through Gibson Assembly method to generate the CD19 CAR-Trap vector.

Trap Protein Purification and Western Blotting Analysis

Using PEIpro (Polyplus) as the transfection reagents, 293T cells were transfected with pcDNA3.1 encoding trap protein labeled with His-tag. Three days after transfection, cell culture supernatant was harvested, and trap protein was purified with HISPUR™ Ni-NTA Resin (Thermo Scientific), following the manufacturer's instruction. The purified trap protein was then quantified using a micro BCA (bicin-choninic acid) protein assay kit (Thermo Scientific).

Twenty ng and 200 ng of purified protein were resolved by SDS polyacrylamide gel electrophoresis (PAGE) and transferred to polyvinylidene fluoride (PVDF) membrane (Bio-Rad). The PVDF membrane was blocked with 5% bovine serum albumin (BSA) for 2 hours and then incubated with anti-6×HIS TAG® antibody-horseradish peroxidase (HRP; 1:10,000 dilution, Abcam) at 4° C. overnight. After incubation, the membrane was washed and developed with Western blotting substrate (Thermo Scientific) and visualized with the enhanced chemiluminescense (ECL) machine (Bio-Rad).

Retroviral Vector Production

Retroviral vectors were prepared by transient transfection of 293T cells using a standard calcium phosphate precipitation method. Briefly, 293T cell were seeded in 15-cm tissue culture dish at a density of 0.8×10⁶ cells/mL. When the confluency reached 70-80%, 293T cells were transfected with 37.5 μg of the retroviral backbone plasmid, together with 18.75 μg of the envelope plasmid RD114 and 30 μg of the packaging plasmid encoding gag-pol. The supernatants were harvested 48 hours after transfection and filtered through a 0.45-μm filter prior to use.

CAR-T Cell Production and Expansion

Frozen human peripheral blood cells (PBMC) were obtained from AllCells. The T cell culture medium (TCM) was composed of AIM-V medium with 5% (vol/vol) human AB serum, 10 mmol/L HEPES, 1% (vol/vol) GlutaMax-100×, 12.25 mmol/L N-acetylcysteine (NAC), 100 U/mL penicillin, and 100 μg/mL streptomycin. The cell culture was supplemented with 10 ng/mL human IL2. After the activation by Dynabeads human T-expander CD3/CD28 (Invitrogen) at a beads: PBMC ratio of 3:1 for 48 hours, PBMCs were transduced with retroviral vectors using the method previously described. Transduced T cells were expanded for 2 weeks in TCM, during which time culture medium was replenished every 2 days and T cell density was maintained between 0.5-1×10⁶ cells/ml.

Flow Cytometry

A MACSQuant Analyzer was used for flow cytometry analysis. Before staining, cells were harvested and washed with fluorescence-activated cell sorting (FACS) buffer (PBS containing 4% bovine serum albumin fraction V). To detect the CAR expression on T cell surface, we used rat anti-mouse biotin conjugated F(ab′)2 antibody (Abcam), followed by allophycocyanin (APC)-conjugated streptavidin (Biolegend). Anti-human PD-L1 antibody and anti-human CD19 antibody (Biolegend) were used to detect the expression of PD-L1 and CD19 on target cell surface, respectively. The following antibodies were used for T cell phenotyping: CD3-fluorescein isothiocyanate (FITC), CD45-phycoerythrin (PE), CD8-Pacific Blue, CD4-PerCP/Cy5.5, PD1-Brilliant Violet 421, PD-L1-PE, TIM3-PE, LAG3-PerCP/Cy5.5, CD107a-APC, CD45RO-PE, CD62L-APC, and CD25-Pacific Blue (Biolegend). For cell surface staining, cells were stained with appropriate antibodies in FACS buffer at 4° C. for 15 min. For intracellular staining, cells were permeabilized by Cytofix/Cytoperm Fixation and Permeabilizaion Solution Kit (BD Biosciences) before being stained with appropriate antibodies in FACS buffer at 4° C. for 30 min. The following antibodies were used for T cell intracellular staining IFN-γ-PE, phospho-Smad2/3-APC, FoxP3-APC, Ki67-Brilliant Violet 421, granzyme B-PE, IL2-APC, and TNF-α-PE. Prior to staining for cells from mouse organs (tumor, spleen, bone marrow, blood), 1×Lysing Solution (BD Biosciences) was used to lyse the red blood cells on ice for 10 min, as recommended by the manufacturer.

ELISA

The TGF-β secretion levels of target cells were evaluated by Human TGF-β1 ELISA Kit (BD Biosciences). Cancer cell lines were seeded at 1×10⁶ cells per well in 6-well tissue culture plate (Corning) for 24 hours, and the supernatants were harvested. The concentration of TGF-β1 in the supernatants was determined by following the manufacturer's protocol of the ELISA kit.

The binding ability of trap protein to PD-1 and TGFβ protein was evaluated by sandwich ELISA, wherein recombinant human PD-1-Fc (GenScript) or TGF-0 protein (R&D Systems) (200 ng/mL) was coated on the plates, followed by purified trap protein (0-40 μg/mL) detected by anti-6×HIS TAG® antibody-HRP (1:500 diluted, Abcam).

IFN-γ secreted by T cells were measured by Human IFN-γ ELISA Set (BD Biosciences). Briefly, 2×10⁵ CAR-T cells were co-cultured with 2×10⁵ PC3-CD19 cells per well in a 96-well round-bottom plate in a 200-μL volume of complete media. Supernatants were harvested at 24 hours, 48 hours and 72 hours after co-culture. The concentration of IFN-γ in the supernatants was determined by following the manufacturer's protocol.

Cytotoxicity Assay

The cytotoxic activity of CAR-T cells against target cells was evaluated by a 24-hour co-culture assay using target cells pre-labeled with CFSE (Invitrogen). We co-cultured NT cells and CAR-T cells with target cells (5×10⁴ cells/well) at effector:target ratios of 1:1, 3:1, and 5:1. After co-culture, 7AAD (BD Biosciences) was added as recommended by the manufacturer. Flow cytometric analysis was performed to quantify the dead target cells (CFSE⁺ and 7-AAD⁺). Cell cytotoxicity was calculated as CFSE⁺7-AAD⁺ cells/(CFSE⁺7-AAD⁻+CFSE⁺7-AAD⁺) cells.

Tumor Model and Adoptive Transfer

The animal experiments were conducted following the animal protocol approved by USC Institutional Animal Care and Use Committee (IACUU). 6 to 8-week-old female NOD.Cg-Prkdc^{scid}IL2Rg^{tm1Wj1}/Sz (NSG) mice (Jackson Laboratory) were inoculated with 3×10⁶ PC3-CD19 cells subcutaneously. When the tumor size reached 75-100 mm³, the mice were randomized into 4 groups and were treated with 2×10⁶ (in the short-term study) or 4×10⁶ (in the long-term study) CAR-T-positive cells in 100 μL of PBS intravenously via tail-vein injection. An equal number of donor-matched non-transduced T (NT) cells were used as a control. CAR expression level was normalized to 20% in all of the CAR-T groups by adding NT cells prior to injection. Tumor growth was monitored every 2 days. Tumor size was calculated as (width×length)/2. For the short-term study, mice were euthanized on day 12 post-treatment, and ex vivo analysis was performed on harvested tumors, spleens, bone marrows and blood. For the long-term study, mice were euthanized when they displayed obvious weight loss, ulceration of tumors, or tumor size larger than 1,000 mm³.

Statistical Analysis

Statistical analysis was performed in GraphPad Prism, version 7.0a. The differences between groups were determined with two-way analysis of variance (ANOVA) with Tukey's multiple comparison. Tumor growth curve was analyzed using one-way ANOVA with repeated measures (Tukey's multiple comparison method). Mice survival curve was evaluated by the Kaplan-Meier analysis. A p value<0.05 was considered statistically significant. Significance of findings was defined as: ns, not significant, p>0.05; *p<0.05; **p<0.01; ***p<0.001.

Polynucleotide sequence of “CD19 CAR-Trap” (SEQ ID NO:9) in the Examples:

(SEQ ID NO: 9)
ATGGCTCTGCCTGTGACCGCCCTGCTGCTGCCTCTGGCTCTGCTGCTGCA
CGCCGCTCGGCCTGACATTCAGATGACTCAGACCACAAGCAGCCTCAGTG
CGAGCCTGGGGGACAGGGTGACTATCAGCTGCCGGGCCAGCCAGGACATT
TCCAAGTACCTGAATTGGTACCAGCAGAAGCCCGATGGTACTGTGAAACT
CCTGATATATCATACTTCTAGGCTCCATTCCGGGGTTCCAAGCCGATTCA
GTGGCTCCGGTTCCGGTACAGATTATTCCCTGACCATTAGCAACTTGGAA
CAGGAGGACATTGCAACGTATTTCTGTCAGCAAGGCAACACATTGCCCTA
CACATTCGGGGGCGGGACTAAACTCGAAATAACTGGCGGCGGGGGTTCTG
GTGGCGGCGGCAGCGGCGGTGGAGGATCAGAAGTGAAGCTGCAGGAAAGT
GGCCCCGGGCTGGTAGCCCCAAGTCAGTCCCTGAGTGTAACCTGTACAGT
GAGTGGAGTGTCTCTTCCTGACTACGGGGTAAGTTGGATTCGGCAACCTC
CACGCAAGGGCCTGGAGTGGCTCGGCGTGATTTGGGGATCTGAGACAACT
TACTACAATTCCGCCCTGAAGAGCAGGCTGACCATCATTAAGGACAATAG
CAAGTCACAGGTGTTTCTGAAGATGAACTCACTGCAGACCGACGACACCG
CCATCTATTACTGCGCCAAACATTATTATTATGGCGGGAGTTATGCTATG
GACTACTGGGGCCAGGGCACTAGCGTCACCGTCAGCAGTACTACAACTCC
AGCACCCAGACCCCCTACACCTGCTCCAACTATCGCAAGTCAGCCCCTGT
CACTGCGCCCTGAAGCCTGTCGCCCTGCTGCCGGGGGAGCTGTGCATACT
CGGGGACTGGACTTTGCCTGTGATATCTACTTCTGGGTGCTGGTCGTGGT
CGGAGGGGTGCTGGCCTGTTATAGCCTGCTGGTGACTGTCGCCTTCATTA
TCTTCTGGGTGCGGAGCAAGAGGTCTCGCGGTGGGCATTCCGACTACATG
AACATGACCCCTAGAAGGCCTGGCCCAACCAGAAAGCACTACCAGCCATA
CGCCCCTCCCAGAGATTTCGCCGCTTATCGAAGCGTGAAGTTCTCCCGAA
GCGCAGATGCCCCAGCCTATCAGCAGGGACAGAATCAGCTGTACAACGAG
CTGAACCTGGGAAGACGGGAGGAATACGATGTGCTGGACAAAAGGCGGGG
CAGAGATCCTGAGATGGGCGGCAAACCAAGACGGAAGAACCCCCAGGAAG
GTCTGTATAATGAGCTGCAGAAAGACAAGATGGCTGAGGCCTACTCAGAA
ATCGGGATGAAGGGCGAAAGAAGGAGAGGAAAAGGCCACGACGGACTGTA
CCAGGGGCTGAGTACAGCAACAAAAGACACCTATGACGCTCTGCACATGC
AGGCTCTGCCACCAAGAAGAGCCAAGCGGGGCTCTGGCGAGGGCAGAGGC
TCTCTGCTGACCTGCGGAGATGTGGAAGAAAATCCCGGCCCTATGTACAG
AATGCAGCTGTTGTCTTGTATTGCCCTTTCTCTCGCCCTCGTAACAAATT
CAGATATAGTACTTACCCAAAGCCCCGCTACATTGAGTTTGTCCCCTGGC
GAGCGGGCCACGCTGTCTTGTCGCGCATCAAAAAGTGTCTCCACATCAGG
ATTCAATTATATGCATTGGTACCAGCAGAAACCTGGGCAAGCGCCAAGGT
TGCTGATTTTCCTTGCCTCCAACCTCGCTTCCGGCGTGCCAGCGCGATTT
TCTGGATCTGGATCAGGAACTGACTTCACTTTGACTATCTCAAGTCTGGA
ACCGGAGGATTTCGCAGTGTATTATTGCCAGCACGGTAGGGAACTGCCTT
TGACATTTGGCCAAGGTACTAAACTCGAGATTGGCGGTGGTGGAAGTGGA
GGAGGGGGATCCGGAGGCGGGGGTTCTCAGGTACAACTGGTACAAAGTGG
CGCAGAAGTCAAAAAACCAGGCGCGAGCGTCAAGGTATCTTGCAAGGCTA
GCGGATACACCTTCACTAACTACTATATGTACTGGGTTCGCCAAGCCCCG
GGTCAAGGCCTGGAATGGATAGGCGGCATAAACCCGTCAAATGGCGGGAC
TAACTTCAATGAAAAATTTAAAAACAAGGCCACAATGACAGTCGATAAAA
GCACTTCCACCGCCTACATGGAACTGTCCTCACTGCGGAGTGAAGACACA
GCCGTTTACTATTGCACCCGACGGGACTACAATTACGACGGTGGGTTCGA
TTACTGGGGTCAGGGAACGTTGGTAACTGTCAGCTCTGGAGGTGGTGGCT
CAGGAGGCGGCGGGAGCACCATTCCCCCTCACGTTCAAAAAAGTGTCAAT
AATGATATGATAGTTACGGATAACAATGGCGCTGTAAAGTTTCCACAGTT
GTGCAAATTCTGTGACGTACGGTTCTCTACCTGTGATAATCAAAAGAGTT
GTATGAGCAATTGCTCTATTACTTCTATTTGTGAAAAACCACAAGAAGTC
TGCGTGGCCGTGTGGCGGAAAAACGATGAGAATATAACCTTGGAGACCGT
GTGTCACGATCCTAAGCTTCCCTATCACGATTTCATACTTGAAGACGCTG
CAAGTCCAAAGTGTATTATGAAGGAAAAAAAGAAACCGGGGGAAACTTTT
TTCATGTGTAGTTGTAGTTCCGATGAGTGTAATGATAACATAATCTTCTC
TGAAGAATATAATACCTCCAATCCAGAT.

The anti-CD19 CAR can be encoded by the following nucleic acid sequence (SEQ ID NO:10):

(SEQ ID NO: 10)
ATGGCTCTGCCTGTGACCGCCCTGCTGCTGCCTCTGGCTCTGCTGCTGCA
CGCCGCTCGGCCTGACATTCAGATGACTCAGACCACAAGCAGCCTCAGTG
CGAGCCTGGGGGACAGGGTGACTATCAGCTGCCGGGCCAGCCAGGACATT
TCCAAGTACCTGAATTGGTACCAGCAGAAGCCCGATGGTACTGTGAAACT
CCTGATATATCATACTTCTAGGCTCCATTCCGGGGTTCCAAGCCGATTCA
GTGGCTCCGGTTCCGGTACAGATTATTCCCTGACCATTAGCAACTTGGAA
CAGGAGGACATTGCAACGTATTTCTGTCAGCAAGGCAACACATTGCCCTA
CACATTCGGGGGCGGGACTAAACTCGAAATAACTGGCGGCGGGGGTTCTG
GTGGCGGCGGCAGCGGCGGTGGAGGATCAGAAGTGAAGCTGCAGGAAAGT
GGCCCCGGGCTGGTAGCCCCAAGTCAGTCCCTGAGTGTAACCTGTACAGT
GAGTGGAGTGTCTCTTCCTGACTACGGGGTAAGTTGGATTCGGCAACCTC
CACGCAAGGGCCTGGAGTGGCTCGGCGTGATTTGGGGATCTGAGACAACT
TACTACAATTCCGCCCTGAAGAGCAGGCTGACCATCATTAAGGACAATAG
CAAGTCACAGGTGTTTCTGAAGATGAACTCACTGCAGACCGACGACACCG
CCATCTATTACTGCGCCAAACATTATTATTATGGCGGGAGTTATGCTATG
GACTACTGGGGCCAGGGCACTAGCGTCACCGTCAGCAGTACTACAACTCC
AGCACCCAGACCCCCTACACCTGCTCCAACTATCGCAAGTCAGCCCCTGT
CACTGCGCCCTGAAGCCTGTCGCCCTGCTGCCGGGGGAGCTGTGCATACT
CGGGGACTGGACTTTGCCTGTGATATCTACTTCTGGGTGCTGGTCGTGGT
CGGAGGGGTGCTGGCCTGTTATAGCCTGCTGGTGACTGTCGCCTTCATTA
TCTTCTGGGTGCGGAGCAAGAGGTCTCGCGGTGGGCATTCCGACTACATG
AACATGACCCCTAGAAGGCCTGGCCCAACCAGAAAGCACTACCAGCCATA
CGCCCCTCCCAGAGATTTCGCCGCTTATCGAAGCGTGAAGTTCTCCCGAA
GCGCAGATGCCCCAGCCTATCAGCAGGGACAGAATCAGCTGTACAACGAG
CTGAACCTGGGAAGACGGGAGGAATACGATGTGCTGGACAAAAGGCGGGG
CAGAGATCCTGAGATGGGCGGCAAACCAAGACGGAAGAACCCCCAGGAAG
GTCTGTATAATGAGCTGCAGAAAGACAAGATGGCTGAGGCCTACTCAGAA
ATCGGGATGAAGGGCGAAAGAAGGAGAGGAAAAGGCCACGACGGACTGTA
CCAGGGGCTGAGTACAGCAACAAAAGACACCTATGACGCTCTGCACATGC
AGGCTCTGCCACCAAGA.

The T2A peptide can be encoded by the following nucleic acid sequence (SEQ ID NO:11):

(SEQ ID NO: 11)
AGAGCCAAGCGGGGCTCTGGCGAGGGCAGAGGCTCTCTGCTGACCTGCGG
AGATGTGGAAGAAAATCCCGGCCCT.

The anti-PD1 scFv (with a human IL-2 leading sequence at the N-terminus) can be encoded by the following nucleic acid sequence (SEQ ID NO:12):

(SEQ ID NO: 12)
ATGTACAGAATGCAGCTGTTGTCTTGTATTGCCCTTTCTCTCGCCCTCGT
AACAAATTCAGATATAGTACTTACCCAAAGCCCCGCTACATTGAGTTTGT
CCCCTGGCGAGCGGGCCACGCTGTCTTGTCGCGCATCAAAAAGTGTCTCC
ACATCAGGATTCAATTATATGCATTGGTACCAGCAGAAACCTGGGCAAGC
GCCAAGGTTGCTGATTTTCCTTGCCTCCAACCTCGCTTCCGGCGTGCCAG
CGCGATTTTCTGGATCTGGATCAGGAACTGACTTCACTTTGACTATCTCA
AGTCTGGAACCGGAGGATTTCGCAGTGTATTATTGCCAGCACGGTAGGGA
ACTGCCTTTGACATTTGGCCAAGGTACTAAACTCGAGATTGGCGGTGGTG
GAAGTGGAGGAGGGGGATCCGGAGGCGGGGGTTCTCAGGTACAACTGGTA
CAAAGTGGCGCAGAAGTCAAAAAACCAGGCGCGAGCGTCAAGGTATCTTG
CAAGGCTAGCGGATACACCTTCACTAACTACTATATGTACTGGGTTCGCC
AAGCCCCGGGTCAAGGCCTGGAATGGATAGGCGGCATAAACCCGTCAAAT
GGCGGGACTAACTTCAATGAAAAATTTAAAAACAAGGCCACAATGACAGT
CGATAAAAGCACTTCCACCGCCTACATGGAACTGTCCTCACTGCGGAGTG
AAGACACAGCCGTTTACTATTGCACCCGACGGGACTACAATTACGACGGT
GGGTTCGATTACTGGGGTCAGGGAACGTTGGTAACTGTCAGCTCT.

The GS linker can be encoded by the following nucleic acid sequence (SEQ ID NO:13):

(SEQ ID NO: 13)
GGAGGTGGTGGCTCAGGAGGCGGCGGGAGC.

The TGFbRII can be encoded by the following nucleic acid sequence (SEQ ID NO:14):

(SEQ ID NO: 14)
ACCATTCCCCCTCACGTTCAAAAAAGTGTCAATAATGATATGATAGTTAC
GGATAACAATGGCGCTGTAAAGTTTCCACAGTTGTGCAAATTCTGTGACG
TACGGTTCTCTACCTGTGATAATCAAAAGAGTTGTATGAGCAATTGCTCT
ATTACTTCTATTTGTGAAAAACCACAAGAAGTCTGCGTGGCCGTGTGGCG
GAAAAACGATGAGAATATAACCTTGGAGACCGTGTGTCACGATCCTAAGC
TTCCCTATCACGATTTCATACTTGAAGACGCTGCAAGTCCAAAGTGTATT
ATGAAGGAAAAAAAGAAACCGGGGGAAACTTTTTTCATGTGTAGTTGTAG
TTCCGATGAGTGTAATGATAACATAATCTTCTCTGAAGAATATAATACCT
CCAATCCAGAT.

Amino acid sequence of “CD19 CAR-Trap” (SEQ ID NO:15) in the Examples:

(SEQ ID NO: 15)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSTTTPAPRPPTPAPTIASQPLSLRPEACRPAAGGAVHT
RGLDFACDIYFWVLVVVGGVLACYSLLVTVAFIIFWVRSKRSRGGHSDYM
NMTPRRPGPTRKHYQPYAPPRDFAAYRSVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPRRAKRGSGEGRG
SLLTCGDVEENPGPMYRMQLLSCIALSLALVTNSDIVLTQSPATLSLSPG
ERATLSCRASKSVSTSGFNYMHWYQQKPGQAPRLLIFLASNLASGVPARF
SGSGSGTDFTLTISSLEPEDFAVYYCQHGRELPLTFGQGTKLEIGGGGSG
GGGSGGGGSQVQLVQSGAEVKKPGASVKVSCKASGYTFTNYYMYWVRQAP
GQGLEWIGGINPSNGGTNFNEKFKNKATMTVDKSTSTAYMELSSLRSEDT
AVYYCTRRDYNYDGGFDYWGQGTLVTVSSGGGGSGGGGSTIPPHVQKSVN
NDMIVTDNNGAVKFPQLCKFCDVRFSTCDNQKSCMSNCSITSICEKPQEV
CVAVWRKNDENITLETVCHDPKLPYHDFILEDAASPKCIMKEKKKPGETF
FMCSCSSDECNDNIIFSEEYNTSNPD.

The anti-CD19 CAR can have an amino acid sequence of SEQ ID NO:16:

(SEQ ID NO: 16)
MALPVTALLLPLALLLHAARPDIQMTQTTSSLSASLGDRVTISCRASQDI
SKYLNWYQQKPDGTVKLLIYHTSRLHSGVPSRFSGSGSGTDYSLTISNLE
QEDIATYFCQQGNTLPYTFGGGTKLEITGGGGSGGGGSGGGGSEVKLQES
GPGLVAPSQSLSVTCTVSGVSLPDYGVSWIRQPPRKGLEWLGVIWGSETT
YYNSALKSRLTIIKDNSKSQVFLKMNSLQTDDTAIYYCAKHYYYGGSYAM
DYWGQGTSVTVSSTTTPAPRPPTPAPTIASQPLSLRPEACRPAAGGAVHT
RGLDFACDIYFWVLVVVGGVLACYSLLVTVAFIIFWVRSKRSRGGHSDYM
NMTPRRPGPTRKHYQPYAPPRDFAAYRSVKFSRSADAPAYQQGQNQLYNE
LNLGRREEYDVLDKRRGRDPEMGGKPRRKNPQEGLYNELQKDKMAEAYSE
IGMKGERRRGKGHDGLYQGLSTATKDTYDALHMQALPPR.

The T2A peptide can have an amino acid sequence of SEQ ID NO:17:

(SEQ ID NO: 17)
RAKRGSGEGRGSLLTCGDVEENPGP.

The anti-PD1 scFv (with a human IL-2 leading sequence at the N-terminus) can have an amino acid sequence of SEQ ID NO:18:

(SEQ ID NO: 18)
MYRMQLLSCIALSLALVTNSDIVLTQSPATLSLSPGERATLSCRASKSVS
TSGFNYMHWYQQKPGQAPRLLIFLASNLASGVPARFSGSGSGTDFTLTIS
SLEPEDFAVYYCQHGRELPLTFGQGTKLEIGGGGSGGGGSGGGGSQVQLV
QSGAEVKKPGASVKVSCKASGYTFTNYYMYWVRQAPGQGLEWIGGINPSN
GGTNFNEKFKNKATMTVDKSTSTAYMELSSLRSEDTAVYYCTRRDYNYDG
GFDYWGQGTLVTVSS.

The GS linker can have an amino acid sequence of SEQ ID NO:19:

(SEQ ID NO: 19)
GGGGSGGGGS.

The TGFbRII can have an amino acid sequence of SEQ ID NO:4:

(SEQ ID NO: 4)
TIPPHVQKSVNNDMIVTDNNGAVKFPQLCKFCDVRFSTCDNQKSCMSNCS
ITSICEKPQEVCVAVWRKNDENITLETVCHDPKLPYHDFILEDAASPKCI
MKEKKKPGETFFMCSCSSDECNDNIIFSEEYNTSNPD.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

What is claimed is:

1. A genetically engineered cell, comprising one or more polynucleotides encoding:
a chimeric antigen receptor (CAR),
a polypeptide that binds an immune checkpoint protein or a polypeptide that binds a ligand of the immune checkpoint protein, and
a polypeptide that binds transforming growth factor beta (TGF-β) or a polypeptide that binds a TGF-β receptor.

2. The genetically engineered cell of claim 1, which expresses the polypeptide that binds an immune checkpoint protein and the polypeptide that binds TGF-β as a fusion protein, and wherein the one or more polynucleotides further encode a signal peptide, located upstream of the 5′ end of the fusion protein.

3. The genetically engineered cell of claim 2, wherein the signal peptide is an interleukin-2 (IL-2) signal peptide or a functional variant thereof.

4. The genetically engineered cell of claim 1, wherein the one or more polynucleotides is one polynucleotide which further encodes a 2A self-cleaving peptide in a configuration wherein the CAR is operably linked via the 2A self-cleaving peptide to 5′ or 3′ end of the polypeptide that binds an immune checkpoint protein or the polypeptide that binds a ligand of the immune checkpoint protein, or to 5′ or 3′ end of the polypeptide that binds TGF-β or the polypeptide that binds a TGF-β receptor, wherein the 2A self-cleaving peptide comprises a T2A peptide, a P2A peptide, an E2A peptide, an F2A peptide, or a combination thereof.

5. The genetically engineered cell of claim 1, wherein the one or more polynucleotides are two polynucleotides, wherein a first of the two polynucleotides encodes the CAR, and a second of the two polypeptides encodes the polypeptide that binds an immune checkpoint protein or the polypeptide that binds a ligand of the immune checkpoint protein and the polypeptide that binds TGF-β or the polypeptide that binds a TGF-β receptor.

6. The genetically engineered cell of claim 1, wherein the polypeptide that binds an immune checkpoint protein comprises a single-chain variable fragment (scFv), a single monomeric variable domain, or an antigen-binding fragment of an anti-programmed cell death protein 1 (PD-1) antibody, an anti-lymphocyte-activation gene 3 (LAG3) antibody, an anti-T cell immunoglobulin domain and mucin domain-containing protein 3 (TIM3) antibody, an anti-T-lymphocyte antigen-4 (CTLA-4) antibody, or a combination thereof; the polypeptide that binds a ligand of the immune checkpoint protein comprises a single-chain variable fragment (scFv), a single monomeric variable domain, or an antigen-binding fragment of an anti-PD-1 ligand (PD-L1) antibody.

7. The genetically engineered cell of claim 1, wherein the one or more polynucleotides encodes the polypeptide that binds an immune checkpoint protein and the polypeptide that binds TGF-β; and
wherein the polypeptide that binds an immune checkpoint protein is a polypeptide that binds PD-1, and the polypeptide that binds PD-1 comprises a single-chain variable fragment (scFv) a single monomeric variable domain, or a PD-1-binding fragment of one or more of nivolumab, pembrolizumab, cemiplimab, dostarlimab, vopratelimab, spartalizumab, camrelizumab, sintilimab, tislelizumab, toripalimab, INCMGA00012, AMP-224, AMP-514, and acrixolimab; and
wherein the polypeptides that binds TGF-β comprises a TGF-β receptor II ectodomain sequence.

8. The genetically engineered cell of claim 1, wherein the cell is a T-lymphocyte (T-cell), a natural killer (NK) cell, a hematopoietic stem cell (HSC), an embryonic stem cell, or a pluripotent stem cell.

9. A composition comprising a plurality of the genetically engineered cells of claim 1, wherein at least 60% of the plurality of the genetically engineered cells express the CAR.

10. The composition of claim 9, wherein at least 20% of the plurality of the genetically engineered cells maintain expression of the CAR after at least one freeze-and-thaw cycle.

11. The composition of claim 9, further comprising a plurality of cells which do not express or secrete a polypeptide inhibitor of an immune checkpoint protein or a polypeptide that binds TGF-β.

12. A polynucleotide, which encodes: (i) a fusion protein comprising a polypeptide checkpoint inhibitor and a polypeptide binder of transforming growth factor beta (TGF-β), and (ii) a chimeric antigen receptor (CAR),
wherein the CAR and the fusion protein are operably linked by a cleavable peptide linker,
wherein the polypeptide checkpoint inhibitor comprises an antigen-binding fragment of one or more of an anti-programmed cell death protein 1 (PD-1) antibody, an anti-lymphocyte-activation gene 3 (LAG3) antibody, an anti-T cell immunoglobulin domain and mucin domain-containing protein 3 (TIM3) antibody, and an anti-T-lymphocyte antigen-4 (CTLA-4) antibody, and
wherein the polypeptide binder of TGF-β comprises a TGF-β-binding fragment of a TGF-β receptor or a TGF-β-binding fragment of an anti-TGF-β antibody.

13. The polynucleotide of claim 12, wherein the cleavable peptide linker comprises a T2A sequence; the polypeptide checkpoint inhibitor comprises a single-chain variable fragment (scFv) of an anti-PD-1 antibody or a fragment thereof being a single-domain antibody, and wherein the polynucleotide from 5′ to 3′ end encodes: the CAR, the T2A sequence, a human IL-2 leading sequence, a light chain variable domain of the anti-PD-1 antibody, a first peptide linker having repeating unit of GGGGS (SEQ ID NO:1), a heavy chain variable domain of the anti-PD-1 antibody, a second peptide linker having repeating unit of GGGGS (SEQ ID NO:1), and the polypeptide binder of TGF-β;
wherein a light chain variable domain and/or a heavy chain variable domain of the anti-PD-1 antibody is derived from one or more of nivolumab, pembrolizumab, cemiplimab, dostarlimab, vopratelimab, spartalizumab, camrelizumab, sintilimab, tislelizumab, toripalimab, INCMGA00012, AMP-224, AMP-514, and acrixolimab; and the polypeptide binder of TGF-β comprises amino acid sequence of a ligand binding region in human TGF-βRII extracellular domain.

14. A vector comprising the polynucleotide of claim 12.

15. A virus comprising the vector of claim 14.

16. A method of generating engineered T-lymphocytes (T-cells) or natural killer (NK) cells, comprising:
transfecting or transducing T-cells or NK cells with the polynucleotide of claim 12, and
expressing the fusion protein in the T-cells or the NK cells.

17. The method of claim 16, further comprising culturing the transfected or transduced T-cells or NK cells in a culture medium and detecting presence of the fusion protein in the culture medium.

18. A method of modifying chimeric antigen receptor (CAR)-expressing immune cells, the method comprising:
transfecting or transducing the CAR-expressing immune cells with a polypeptide encoding a fusion protein comprising a polypeptide checkpoint inhibitor and a polypeptide binder of transforming growth factor beta (TGF-β), so as for the CAR-expressing immune cells to express the fusion protein.

19. A method of treating a subject having a tumor, having undergone an anti-cancer therapy, or in need of inhibiting a tumor relapse, comprising administering to the subject an effective amount of the genetically engineered cell of claim 1.

20. A method of treating a subject having a tumor, having undergone an anti-cancer therapy, or in need of inhibiting a tumor relapse, comprising administering to the subject the engineered T-cells generated by the method of claim 16.


SEQUENCE LISTING (22 sequences)

SEQ ID NO: 1 - protein, 5 aa, synthetic construct
GGGGS

SEQ ID NO: 2 - protein, 18 aa, synthetic construct
KESGSVSSEQLAQFRSLD

SEQ ID NO: 3 - protein, 14 aa, synthetic construct
EGKSSGSGSESKST

SEQ ID NO: 4 - protein, 137 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 5 - protein, 18 aa, Thosea asigna virus
EGRGSLLTCGDVEENPGP

SEQ ID NO: 6 - protein, 19 aa, Teschovirus A
ATNFSLLKQAGDVEENPGP

SEQ ID NO: 7 - protein, 20 aa, Equine rhinitis A virus
QCTNYALLKLAGDVESNPGP

SEQ ID NO: 8 - protein, 22 aa, Foot-and-mouth disease virus
VKQTLNFDLLKLAGDVESNPGP

SEQ ID NO: 9 - DNA, 2778 bp, synthetic construct
  source 1..2778: mol_type = other DNA
atggctctgcctgtgaccgccctgctgctgcctctggctctgctgctgcacgccgctcggcctgacattcagatgactcagaccacaagcagcctcagtgcgagcctgggggacagggtgactatcagctgccgggccagccaggacatttccaagtacctgaattggtaccagcagaagcccgatggtactgtgaaactcctgatatatcatacttctaggctccattccggggttccaagccgattcagtggctccggttccggtacagattattccctgaccattagcaacttggaacaggaggacattgcaacgtatttctgtcagcaaggcaacacattgccctacacattcgggggcgggactaaactcgaaataactggcggcgggggttctggtggcggcggcagcggcggtggaggatcagaagtgaagctgcaggaaagtggccccgggctggtagccccaagtcagtccctgagtgtaacctgtacagtgagtggagtgtctcttcctgactacggggtaagttggattcggcaacctccacgcaagggcctggagtggctcggcgtgatttggggatctgagacaacttactacaattccgccctgaagagcaggctgaccatcattaaggacaatagcaagtcacaggtgtttctgaagatgaactcactgcagaccgacgacaccgccatctattactgcgccaaacattattattatggcgggagttatgctatggactactggggccagggcactagcgtcaccgtcagcagtactacaactccagcacccagaccccctacacctgctccaactatcgcaagtcagcccctgtcactgcgccctgaagcctgtcgccctgctgccgggggagctgtgcatactcggggactggactttgcctgtgatatctacttctgggtgctggtcgtggtcggaggggtgctggcctgttatagcctgctggtgactgtcgccttcattatcttctgggtgcggagcaagaggtctcgcggtgggcattccgactacatgaacatgacccctagaaggcctggcccaaccagaaagcactaccagccatacgcccctcccagagatttcgccgcttatcgaagcgtgaagttctcccgaagcgcagatgccccagcctatcagcagggacagaatcagctgtacaacgagctgaacctgggaagacgggaggaatacgatgtgctggacaaaaggcggggcagagatcctgagatgggcggcaaaccaagacggaagaacccccaggaaggtctgtataatgagctgcagaaagacaagatggctgaggcctactcagaaatcgggatgaagggcgaaagaaggagaggaaaaggccacgacggactgtaccaggggctgagtacagcaacaaaagacacctatgacgctctgcacatgcaggctctgccaccaagaagagccaagcggggctctggcgagggcagaggctctctgctgacctgcggagatgtggaagaaaatcccggccctatgtacagaatgcagctgttgtcttgtattgccctttctctcgccctcgtaacaaattcagatatagtacttacccaaagccccgctacattgagtttgtcccctggcgagcgggccacgctgtcttgtcgcgcatcaaaaagtgtctccacatcaggattcaattatatgcattggtaccagcagaaacctgggcaagcgccaaggttgctgattttccttgcctccaacctcgcttccggcgtgccagcgcgattttctggatctggatcaggaactgacttcactttgactatctcaagtctggaaccggaggatttcgcagtgtattattgccagcacggtagggaactgcctttgacatttggccaaggtactaaactcgagattggcggtggtggaagtggaggagggggatccggaggcgggggttctcaggtacaactggtacaaagtggcgcagaagtcaaaaaaccaggcgcgagcgtcaaggtatcttgcaaggctagcggatacaccttcactaactactatatgtactgggttcgccaagccccgggtcaaggcctggaatggataggcggcataaacccgtcaaatggcgggactaacttcaatgaaaaatttaaaaacaaggccacaatgacagtcgataaaagcacttccaccgcctacatggaactgtcctcactgcggagtgaagacacagccgtttactattgcacccgacgggactacaattacgacggtgggttcgattactggggtcagggaacgttggtaactgtcagctctggaggtggtggctcaggaggcggcgggagcaccattccccctcacgttcaaaaaagtgtcaataatgatatgatagttacggataacaatggcgctgtaaagtttccacagttgtgcaaattctgtgacgtacggttctctacctgtgataatcaaaagagttgtatgagcaattgctctattacttctatttgtgaaaaaccacaagaagtctgcgtggccgtgtggcggaaaaacgatgagaatataaccttggagaccgtgtgtcacgatcctaagcttccctatcacgatttcatacttgaagacgctgcaagtccaaagtgtattatgaaggaaaaaaagaaaccgggggaaacttttttcatgtgtagttgtagttccgatgagtgtaatgataacataatcttctctgaagaatataatacctccaatccagat

SEQ ID NO: 10 - DNA, 1467 bp, synthetic construct
  source 1..1467: mol_type = other DNA
atggctctgcctgtgaccgccctgctgctgcctctggctctgctgctgcacgccgctcggcctgacattcagatgactcagaccacaagcagcctcagtgcgagcctgggggacagggtgactatcagctgccgggccagccaggacatttccaagtacctgaattggtaccagcagaagcccgatggtactgtgaaactcctgatatatcatacttctaggctccattccggggttccaagccgattcagtggctccggttccggtacagattattccctgaccattagcaacttggaacaggaggacattgcaacgtatttctgtcagcaaggcaacacattgccctacacattcgggggcgggactaaactcgaaataactggcggcgggggttctggtggcggcggcagcggcggtggaggatcagaagtgaagctgcaggaaagtggccccgggctggtagccccaagtcagtccctgagtgtaacctgtacagtgagtggagtgtctcttcctgactacggggtaagttggattcggcaacctccacgcaagggcctggagtggctcggcgtgatttggggatctgagacaacttactacaattccgccctgaagagcaggctgaccatcattaaggacaatagcaagtcacaggtgtttctgaagatgaactcactgcagaccgacgacaccgccatctattactgcgccaaacattattattatggcgggagttatgctatggactactggggccagggcactagcgtcaccgtcagcagtactacaactccagcacccagaccccctacacctgctccaactatcgcaagtcagcccctgtcactgcgccctgaagcctgtcgccctgctgccgggggagctgtgcatactcggggactggactttgcctgtgatatctacttctgggtgctggtcgtggtcggaggggtgctggcctgttatagcctgctggtgactgtcgccttcattatcttctgggtgcggagcaagaggtctcgcggtgggcattccgactacatgaacatgacccctagaaggcctggcccaaccagaaagcactaccagccatacgcccctcccagagatttcgccgcttatcgaagcgtgaagttctcccgaagcgcagatgccccagcctatcagcagggacagaatcagctgtacaacgagctgaacctgggaagacgggaggaatacgatgtgctggacaaaaggcggggcagagatcctgagatgggcggcaaaccaagacggaagaacccccaggaaggtctgtataatgagctgcagaaagacaagatggctgaggcctactcagaaatcgggatgaagggcgaaagaaggagaggaaaaggccacgacggactgtaccaggggctgagtacagcaacaaaagacacctatgacgctctgcacatgcaggctctgccaccaaga

SEQ ID NO: 11 - DNA, 75 bp, synthetic construct
  source 1..75: mol_type = other DNA
agagccaagcggggctctggcgagggcagaggctctctgctgacctgcggagatgtggaagaaaatcccggccct

SEQ ID NO: 12 - DNA, 795 bp, synthetic construct
  source 1..795: mol_type = other DNA
atgtacagaatgcagctgttgtcttgtattgccctttctctcgccctcgtaacaaattcagatatagtacttacccaaagccccgctacattgagtttgtcccctggcgagcgggccacgctgtcttgtcgcgcatcaaaaagtgtctccacatcaggattcaattatatgcattggtaccagcagaaacctgggcaagcgccaaggttgctgattttccttgcctccaacctcgcttccggcgtgccagcgcgattttctggatctggatcaggaactgacttcactttgactatctcaagtctggaaccggaggatttcgcagtgtattattgccagcacggtagggaactgcctttgacatttggccaaggtactaaactcgagattggcggtggtggaagtggaggagggggatccggaggcgggggttctcaggtacaactggtacaaagtggcgcagaagtcaaaaaaccaggcgcgagcgtcaaggtatcttgcaaggctagcggatacaccttcactaactactatatgtactgggttcgccaagccccgggtcaaggcctggaatggataggcggcataaacccgtcaaatggcgggactaacttcaatgaaaaatttaaaaacaaggccacaatgacagtcgataaaagcacttccaccgcctacatggaactgtcctcactgcggagtgaagacacagccgtttactattgcacccgacgggactacaattacgacggtgggttcgattactggggtcagggaacgttggtaactgtcagctct

SEQ ID NO: 13 - DNA, 30 bp, synthetic construct
  source 1..30: mol_type = other DNA
ggaggtggtggctcaggaggcggcgggagc

SEQ ID NO: 14 - DNA, 411 bp, synthetic construct
  source 1..411: mol_type = other DNA
accattccccctcacgttcaaaaaagtgtcaataatgatatgatagttacggataacaatggcgctgtaaagtttccacagttgtgcaaattctgtgacgtacggttctctacctgtgataatcaaaagagttgtatgagcaattgctctattacttctatttgtgaaaaaccacaagaagtctgcgtggccgtgtggcggaaaaacgatgagaatataaccttggagaccgtgtgtcacgatcctaagcttccctatcacgatttcatacttgaagacgctgcaagtccaaagtgtattatgaaggaaaaaaagaaaccgggggaaacttttttcatgtgtagttgtagttccgatgagtgtaatgataacataatcttctctgaagaatataatacctccaatccagat

SEQ ID NO: 15 - protein, 926 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYFWVLVVVGGVLACYSLLVTVAFIIFWVRSKRSRGGHSDYMNMTPRRPGPTRKHYQPYAPPRDFAAYRSVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGEGRGSLLTCGDVEENPGPMYRMQLLSCIALSLALVTNSDIVLTQSPATLSLSPGERATLSCRASKSVSTSGFNYMHWYQQKPGQAPRLLIFLASNLASGVPARFSGSGSGTDFTLTISSLEPEDFAVYYCQHGRELPLTFGQGTKLEIGGGGSGGGGSGGGGSQVQLVQSGAEVKKPGASVKVSCKASGYTFTNYYMYWVRQAPGQGLEWIGGINPSNGGTNFNEKFKNKATMTVDKSTSTAYMELSSLRSEDTAVYYCTRRDYNYDGGFDYWGQGTLVTVSSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 16 - protein, 489 aa, synthetic construct
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYFWVLVVVGGVLACYSLLVTVAFIIFWVRSKRSRGGHSDYMNMTPRRPGPTRKHYQPYAPPRDFAAYRSVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 25 aa, synthetic construct
RAKRGSGEGRGSLLTCGDVEENPGP

SEQ ID NO: 18 - protein, 265 aa, synthetic construct
MYRMQLLSCIALSLALVTNSDIVLTQSPATLSLSPGERATLSCRASKSVSTSGFNYMHWYQQKPGQAPRLLIFLASNLASGVPARFSGSGSGTDFTLTISSLEPEDFAVYYCQHGRELPLTFGQGTKLEIGGGGSGGGGSGGGGSQVQLVQSGAEVKKPGASVKVSCKASGYTFTNYYMYWVRQAPGQGLEWIGGINPSNGGTNFNEKFKNKATMTVDKSTSTAYMELSSLRSEDTAVYYCTRRDYNYDGGFDYWGQGTLVTVSS

SEQ ID NO: 19 - protein, 10 aa, synthetic construct
GGGGSGGGGS

SEQ ID NO: 20 - protein, 15 aa, synthetic construct
GGGGSGGGGSGGGGS

SEQ ID NO: 21 - protein, 8 aa, unidentified
  VARIANT 2: note = X can be V or I
  VARIANT 4: note = X can be any amino acid
DXEXNPGP

SEQ ID NO: 22 - protein, 20 aa, Homo sapiens
MYRMQLLSCIALSLALVTNS